SEQUENCE LISTING (45 sequences)

SEQ ID NO: 1 - DNA, 1887 bp, Artificial Sequence
  MDP- 20-127 - Protein D - PRAME - TSGHHHHHH (plasmid TCMP14)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaacactagtggccaccatcaccatcaccat

SEQ ID NO: 2 - protein, 629 aa, Artificial Sequence [sequence omitted]

SEQ ID NO: 3 - DNA, 1860 bp, Artificial Sequence
  MDP- 20-127 - Protein D - PRAME - no His tail (plasmid TCMP14)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaac

SEQ ID NO: 4 - protein, 620 aa, Artificial Sequence
  MDP- 20-127 - Protein D - PRAME - no His tail (plasmid TCMP14)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 5 - DNA, 1884 bp, Artificial Sequence
  MDP- 20-127 - Protein D - PRAME - LEHHHHHH (plasmid pET21)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacaaccttaagcttctacgggaattccatctccatatctgccttgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaacctcgagcaccaccaccaccaccac

SEQ ID NO: 6 - protein, 628 aa, Artificial Sequence
  MDP- 20-127 - Protein D - PRAME - LEHHHHHH (plasmid pET21)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPNLEHHHHHH

SEQ ID NO: 7 - DNA, 1860 bp, Artificial Sequence; identical to SEQ ID NO: 3
  MDP- 20-127 - Protein D - PRAME - no His tail (plasmid pET21)

SEQ ID NO: 8 - protein, 620 aa, Artificial Sequence; identical to SEQ ID NO: 4
  MDP- 20-127 - Protein D - PRAME - no His tail (plasmid pET21)

SEQ ID NO: 9 - DNA, 1878 bp, Artificial Sequence
  MDP- 20-127 - Protein D - PRAME - HHHHHH (plasmid pET26)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggaacgtcgtcgtctgtggggcagcattcagagccgttatattagcatgagcgtgtggaccagcccgcgtcgtctggttgagctggccggccagagcctgctgaaagatgaagcgctggccattgcggcgctggagctgctgccgcgtgagctgtttccgccgctgtttatggcggcgtttgatggccgtcatagccagaccctgaaagcgatggtgcaggcgtggccgtttacctgtctgccgctgggcgtgctgatgaaaggccagcatctgcatctggaaacctttaaagcggtgctggatggcctggatgtgctgctggcccaggaagttcgtccgcgtcgttggaaactgcaagtgctggatctgcgtaaaaacagccatcaggatttttggaccgtgtggagcggcaatcgtgcgagcctgtatagctttccggaaccggaagcggcgcagccgatgaccaaaaaacgtaaagtggatggcctgagcaccgaagcggaacagccgtttattccggtggaagtgctggttgacctgtttctgaaagaaggcgcctgcgacgagctgtttagctatctgatcgaaaaagtgaaacgcaaaaaaaacgtgctgcgtctgtgctgcaaaaaactgaaaatcttcgcgatgccgatgcaggatattaaaatgatcctgaaaatggtgcagctggatagcattgaggacctggaagtgacctgcacctggaaactgccgaccctggccaaatttagcccgtatctgggccagatgattaacctgcgtcgtctgctgctgtctcatattcatgcgagcagctatattagcccggaaaaagaagaacagtatatcgcgcagtttaccagccagtttctgagcctgcaatgcctgcaagcgctgtatgtggatagcctgttttttctgcgtggccgtctggatcagctgctgcgtcatgtgatgaatccgctggaaaccctgagcattaccaactgccgtctgagcgaaggcgatgtgatgcatctgagccagagcccgagcgttagccagctgtctgttctgagcctgagcggcgtgatgctgaccgatgtgagcccggaaccgctgcaagccctgctggaacgtgcgagcgcgaccctgcaagacctggtgtttgatgaatgcggcattaccgatgatcagctgctggccctgctgccgagcctgagccattgcagccagctgaccaccctgagcttttatggcaacagcattagcattagcgcgctgcaaagcctgctgcaacatctgattggcctgagcaacctgacccatgtgctgtatccggtgccgctggaaagctatgaagatattcatggcaccctgcatctggaacgtctggcctatctgcacgcgcgtctgcgtgagctgctgtgcgagctgggccgtccgagcatggtttggctgtctgcgaatccgtgcccgcattgcggcgatcgtaccttttatgatccggaaccgattctgtgcccgtgctttatgccgaaccaccaccaccaccaccac

SEQ ID NO: 10 - protein, 626 aa, Artificial Sequence
  MDP- 20-127 - Protein D - PRAME - HHHHHH (plasmid pET26)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPNHHHHHH

SEQ ID NO: 11 - DNA, 1860 bp, Artificial Sequence
  MDP- 20-127 - Protein D - PRAME - no His tail (plasmid pET26)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggaacgtcgtcgtctgtggggcagcattcagagccgttatattagcatgagcgtgtggaccagcccgcgtcgtctggttgagctggccggccagagcctgctgaaagatgaagcgctggccattgcggcgctggagctgctgccgcgtgagctgtttccgccgctgtttatggcggcgtttgatggccgtcatagccagaccctgaaagcgatggtgcaggcgtggccgtttacctgtctgccgctgggcgtgctgatgaaaggccagcatctgcatctggaaacctttaaagcggtgctggatggcctggatgtgctgctggcccaggaagttcgtccgcgtcgttggaaactgcaagtgctggatctgcgtaaaaacagccatcaggatttttggaccgtgtggagcggcaatcgtgcgagcctgtatagctttccggaaccggaagcggcgcagccgatgaccaaaaaacgtaaagtggatggcctgagcaccgaagcggaacagccgtttattccggtggaagtgctggttgacctgtttctgaaagaaggcgcctgcgacgagctgtttagctatctgatcgaaaaagtgaaacgcaaaaaaaacgtgctgcgtctgtgctgcaaaaaactgaaaatcttcgcgatgccgatgcaggatattaaaatgatcctgaaaatggtgcagctggatagcattgaggacctggaagtgacctgcacctggaaactgccgaccctggccaaatttagcccgtatctgggccagatgattaacctgcgtcgtctgctgctgtctcatattcatgcgagcagctatattagcccggaaaaagaagaacagtatatcgcgcagtttaccagccagtttctgagcctgcaatgcctgcaagcgctgtatgtggatagcctgttttttctgcgtggccgtctggatcagctgctgcgtcatgtgatgaatccgctggaaaccctgagcattaccaactgccgtctgagcgaaggcgatgtgatgcatctgagccagagcccgagcgttagccagctgtctgttctgagcctgagcggcgtgatgctgaccgatgtgagcccggaaccgctgcaagccctgctggaacgtgcgagcgcgaccctgcaagacctggtgtttgatgaatgcggcattaccgatgatcagctgctggccctgctgccgagcctgagccattgcagccagctgaccaccctgagcttttatggcaacagcattagcattagcgcgctgcaaagcctgctgcaacatctgattggcctgagcaacctgacccatgtgctgtatccggtgccgctggaaagctatgaagatattcatggcaccctgcatctggaacgtctggcctatctgcacgcgcgtctgcgtgagctgctgtgcgagctgggccgtccgagcatggtttggctgtctgcgaatccgtgcccgcattgcggcgatcgtaccttttatgatccggaaccgattctgtgcccgtgctttatgccgaac

SEQ ID NO: 12 - protein, 620 aa, Artificial Sequence; identical to SEQ ID NO: 4
  MDP- 20-127 - Protein D - PRAME - no His tail (plasmid pET26)

SEQ ID NO: 13 - protein, 9 aa, Homo sapiens
VLDGLDVLL

SEQ ID NO: 14 - protein, 10 aa, Homo sapiens
SLYSFPEPEA

SEQ ID NO: 15 - protein, 10 aa, Homo sapiens
ALYVDSLFFL

SEQ ID NO: 16 - protein, 9 aa, Homo sapiens
LYVDSLFFL

SEQ ID NO: 17 - protein, 9 aa, Homo sapiens
SLLQHLIGL

SEQ ID NO: 18 - DNA, 36 bp, Artificial Sequence
  sense oligonucleotide CAN008
atataacatatggatccaagcagccattcatcaaat

SEQ ID NO: 19 - DNA, 40 bp, Artificial Sequence
  antisense oligonucleotide CAN037
ccacaaacgccttcgttccatggtttcaaagttttctgtc

SEQ ID NO: 20 - DNA, 40 bp, Artificial Sequence
  sense oligonucleotide CAN036
gacagaaaactttgaaaccatggaacgaaggcgtttgtgg

SEQ ID NO: 21 - DNA, 39 bp, Artificial Sequence
  antisense oligonucleotide CAN029
agagagactagtctagttaggcatgaaacaggggcacag

SEQ ID NO: 22 - DNA, 36 bp, Artificial Sequence
  antisense oligonucleotide CAN002
ggaggaactagtgttaggcatgaaacaggggcacag

SEQ ID NO: 23 - DNA, 36 bp, Artificial Sequence
  sense oligonucleotide CAN040
agagagcatatgagcagccattcatcaaatatggcg

SEQ ID NO: 24 - DNA, 41 bp, Artificial Sequence
  antisense oligonucleotide CAN032
acgtgggcggccgcggtttcaaagttttctgtcatttctaa

SEQ ID NO: 25 - DNA, 39 bp, Artificial Sequence
  sense oligonucleotide CAN033
ttgttggcggccgcaatggaacgaaggcgtttgtggggt

SEQ ID NO: 26 - DNA, 36 bp, Artificial Sequence
  antisense oligonucleotide CAN034
ggaggactcgaggttaggcatgaaacaggggcacag

SEQ ID NO: 27 - DNA, 39 bp, Artificial Sequence
  antisense oligonucleotide CAN035
ggaggactcgagctagttaggcatgaaacaggggcacag

SEQ ID NO: 28 - DNA, 31 bp, Artificial Sequence
  sense oligonucleotide CAN106
cagaaaactttgaaaccatggaacgaaggcg

SEQ ID NO: 29 - DNA, 31 bp, Artificial Sequence
  antisense oligonucleotide CAN107
cgccttcgttccatggtttcaaagttttctg

SEQ ID NO: 30 - DNA, 43 bp, Artificial Sequence
  sense oligonucleotide CAN104
ggagatatacatatggatccaagcagccattcatcaaatatgg

SEQ ID NO: 31 - DNA, 43 bp, Artificial Sequence
  antisense oligonucleotide CAN105
ccatatttgatgaatggctgcttggatccatatgtatatctcc

SEQ ID NO: 32 - DNA, 40 bp, Artificial Sequence
  sense oligonucleotide CAN123
gacagaaaactttgaaaccatggaacgtcgtcgtctgtgg

SEQ ID NO: 33 - DNA, 40 bp, Artificial Sequence
  antisense oligonucleotide CAN124
ccacagacgacgacgttccatggtttcaaagttttctgtc

SEQ ID NO: 34 - DNA, 30 bp, Artificial Sequence
  sense oligonucleotide CAN199
ggaattccatatggatccaagcagccattc

SEQ ID NO: 35 - DNA, 53 bp, Artificial Sequence
  antisense oligonucleotide CAN19
ggagctctcgagtcagtggtggtggtggtggtggttcggcataaagcacgggc

SEQ ID NO: 36 - DNA, 20 bp, Artificial Sequence
  immunostimulatory oligonucleotide CpG 1826
tccatgacgttcctgacgtt

SEQ ID NO: 37 - DNA, 18 bp, Artificial Sequence
  immunostimulatory oligonucleotide CpG 1758
tctcccagcgtgcgccat

SEQ ID NO: 38 - DNA, 54 bp, Artificial Sequence
  immunostimulatory oligonucleotide CpG2006
accgatgacgtcgccggtgacggcaccacgtcgtcgttttgtcgttttgtcgtt

SEQ ID NO: 39 - DNA, 20 bp, Artificial Sequence
  immunostimulatory oligonucleotide CpG1668
tccatgacgttcctgatgct

SEQ ID NO: 40 - DNA, 22 bp, Artificial Sequence
  immunostimulatory oligonucleotide CpG5456
tcgacgttttcggcgcgcgccg

SEQ ID NO: 41 - protein, 127 aa, Haemophilus influenzae b
MKLKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFET

SEQ ID NO: 42 - protein, 111 aa, Artificial Sequence
  1-Met; 2-Asp; 3-Pro; followed by AA 20 to 127 of Protein D
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFET

SEQ ID NO: 43 - protein, 450 aa, Artificial Sequence
  Protein D-MAGE-A3-His
MDPKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMDLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGHHHHHHH

SEQ ID NO: 44 - protein, 109 aa, Artificial Sequence
  the aa from protein D from Haemophilus influenzae for use in a pD1/3-PRAME-His sequence
MSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFET

SEQ ID NO: 45 - protein, 127 aa, Artificial Sequence
  the aa from protein D from Haemophilus influenzae for use in a pD1/3-MAGE-His sequence
MDPKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFET

(19) United States
(12) Patent Application Publication
(10) Pub. No.: US 2008/0187535 A1
(43) Pub. Date: Aug. 7, 2008

(54) VACCINE

(76) Inventors: Normand Blais, Laval (CA); Denis Martin, Laval (CA); Remi M. Palmantier, Laval (CA)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/013,011

(22) Filed: Jan. 11, 2008

(30) Foreign Application Priority Data
Jan. 15, 2007 (GB) 0700760.2
Jan. 23, 2007 (GB) 0701262.8

(51) Int. Cl. A61K 39/00; C07K 16/00; C12N 15/00; C12P 21/04; C07H 21/04

(52) U.S. Cl. 424/134.1; 530/387.3; 536/23.4; 435/471; 435/69.7

(57) ABSTRACT

The present invention relates to fusion proteins comprising an antigen derived from the so-called tumour rejection antigen PRAME (also known as DAGE) linked to an immunological fusion partner which provides T helper epitopes, such as, for example protein D from Haemophilus influenzae B, fusion partner proteins comprising fragments of protein D, methods for preparing the same and for formulating vaccines and use of the same for treating a range of cancers.

The present invention relates to fusion proteins comprising an antigen derived from the so-called tumour rejection antigen PRAME (also known as DAGE) linked to an immunological fusion partner which provides T helper epitopes, such as, for example protein D from Haemophilus influenzae B, methods for preparing the same and for formulating vaccines and use of the same for treating a range of cancers, including, but not limited to melanoma, breast, bladder, lung cancer such as NSCLC, sarcoma, ovarian cancer, head and neck cancer, renal cancer, colorectal carcinoma, multiple myeloma, leukemia including acute leukemia and oesophageal carcinoma.

In a further embodiment, the present invention relates to fusion partner proteins comprising protein D derivatives and methods for preparing same.

Among different groups of tumour-associated antigens, cancer testis antigens are of interest for immunotherapy because of their broad tumour-specific expression and the fact that generally these antigens are not expressed in healthy cells. More than 50 cancer/testis antigens have been described so far and, for many of them, epitopes recognized by T lymphocytes have been identified. PRAME is a cancer testis antigen and is in under investigation as a potential immunotherapy.

In immunotherapy the cancer antigen is introduced to the patient usually as a vaccine, for example containing an antigen as a protein or an immunogenic fragment thereof, or as DNA encoding for the protein or as a vector containing said DNA, which stimulates the patient's immune system to attack tumours expressing the same antigen.

If the appropriate response is stimulated, T lymphocytes (T cells) attack antigens directly, and provide control of the immune response. B cells and T cells develop that are specific for one antigen type. When the immune system is exposed to a different antigen, different B cells and T cells are formed. As lymphocytes develop, they normally learn to recognize the body's own tissues (self) as different from tissues and particles not normally found in the body (non-self). Once B cells and T cells are formed, a few of those cells will multiply and provide “memory” for the immune system. This allows the immune system to respond faster and more efficiently the next time it is exposed to the same antigen.

Certain experiments seem to indicate that cancer testis antigens can stimulate the memory mechanisms in the immune system.

It is hypothesized by some that PRAME is involved in cell death or cell cycles. It has been shown by some groups to be expressed in melanoma and a wide variety of tumours including lung, kidney and head and neck. Interestingly it also seems to be expressed in 40-60% leukemia such as acute lymphoid leukemia and acute myeloid leukemia, see for example Exp Hematol. December 2000;28(12):1413-22. In patients it has been observed that over expression of PRAME seems to be associated with higher survival and lower rates of relapse in comparison to those who do not over express the protein.

The antigen and its preparation are described in U.S. Pat. No. 5,830,753. PRAME is found in the Annotated Human Gene Database H-Inv DB under the accession numbers: U65011.1, BC022008.1, AK129783.1, BC014974.2, CR608334.1, AF025440.1, CR591755.1, BC039731.1, CR623010.1, CR611321.1, CR618501.1, CR604772.1, CR456549.1, and CR620272.1.

Protein D is a surface protein of the gram-negative bacterium, Haemophilus influenza B. Information on immunological fusion partners derived from protein D can be obtained from WO 91/18926.

Fusion proteins of a portion of an antigen and a heterologous fusion partner are sometimes prepared to increase the immunogenicity of the antigen and/or aid production of the protein in appropriate quantities and/or purity see for example WO 99/40188 which describes a fusion protein of MAGE and, for example protein D a surface protein of the gram-negative bacterium, Haemophilus influenza B. The fusion protein is prepared recombinantly and the protein D secretion sequence can be incorporated into the fusion protein to potentially assist secretion and solubilization of the final product.

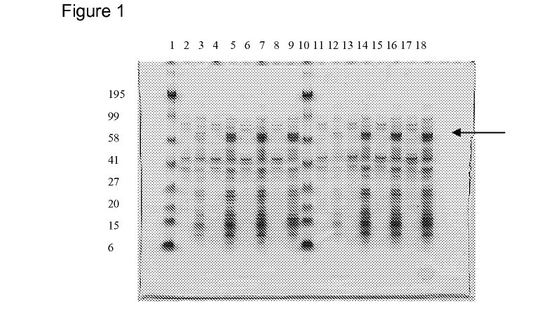

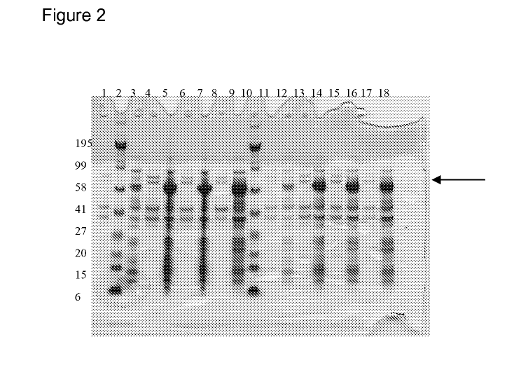

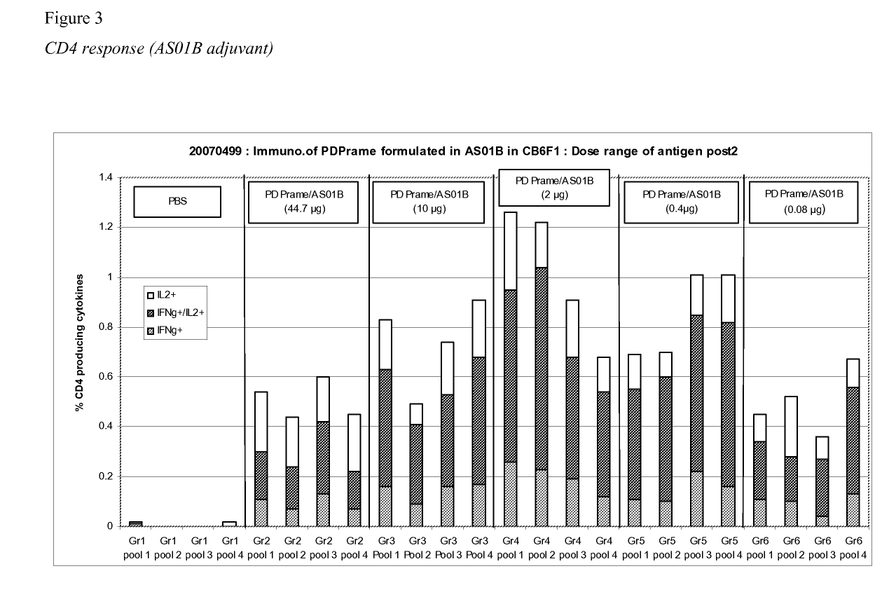

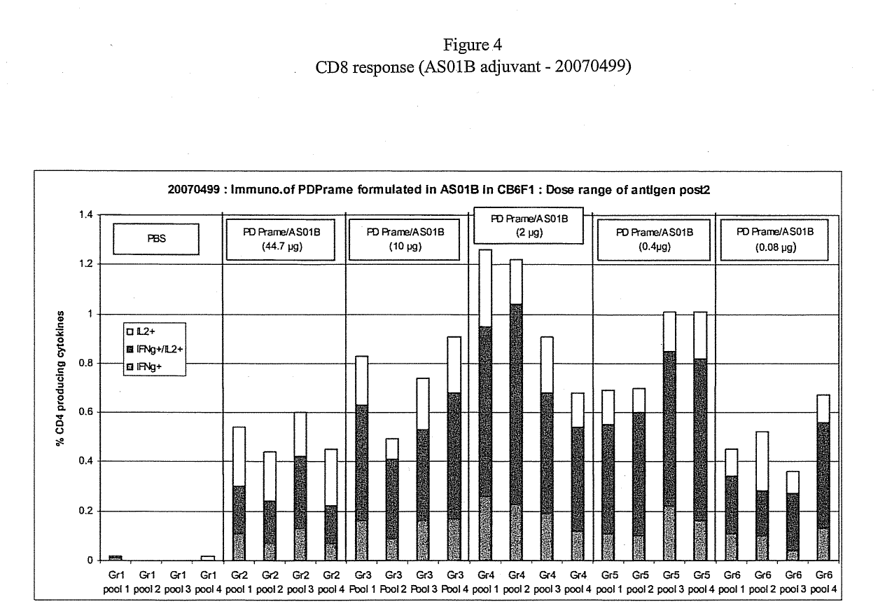

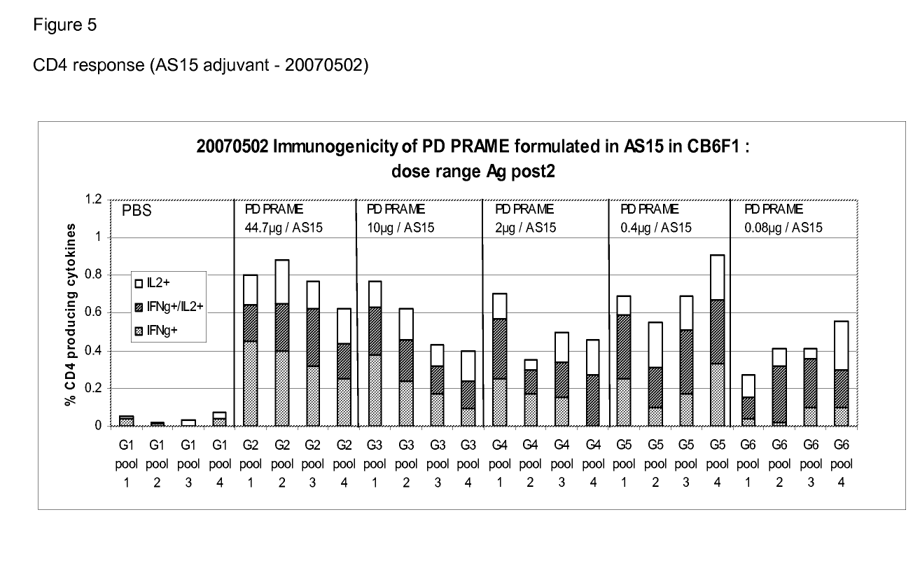

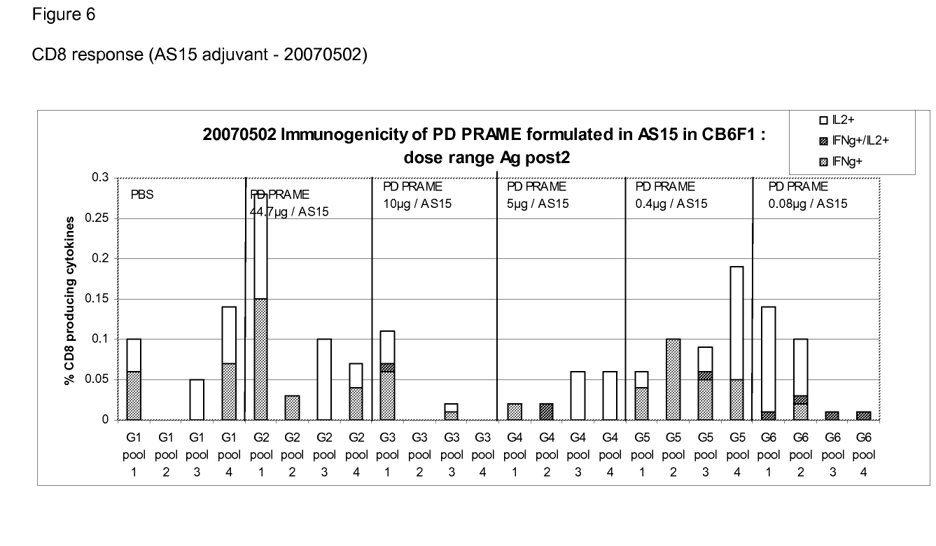

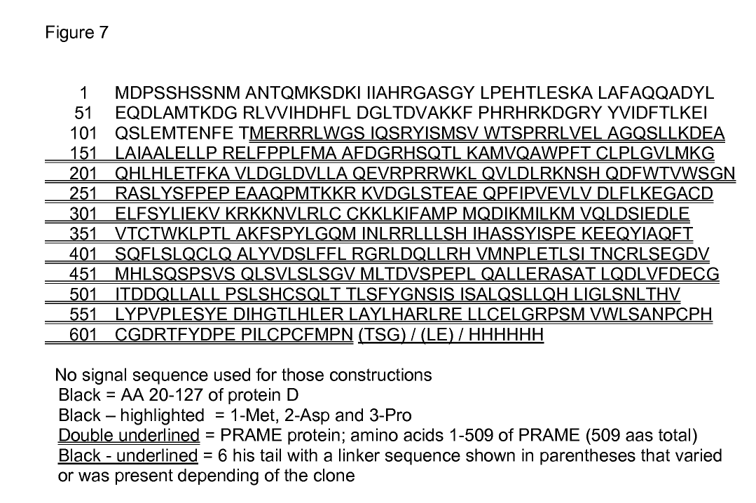

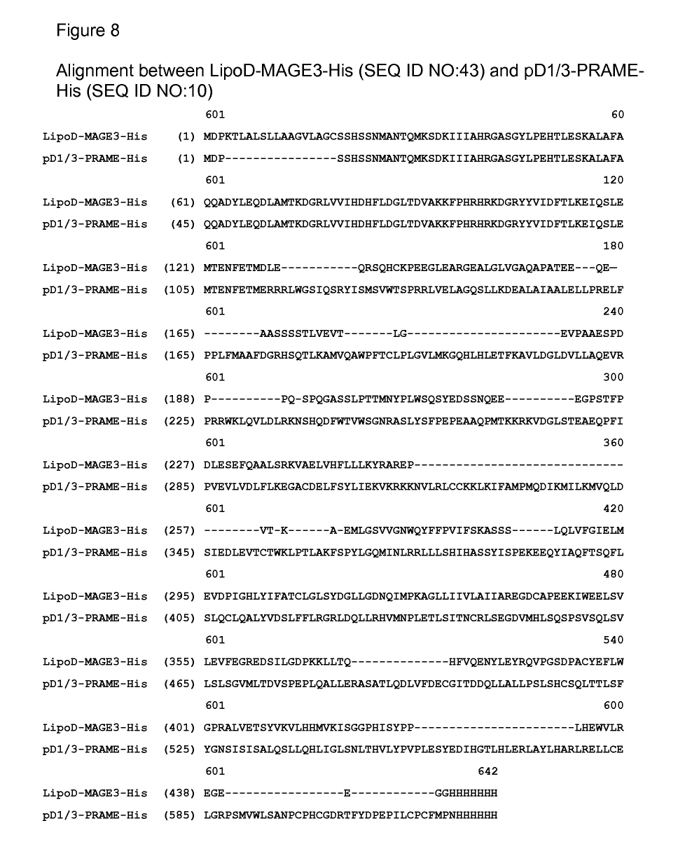

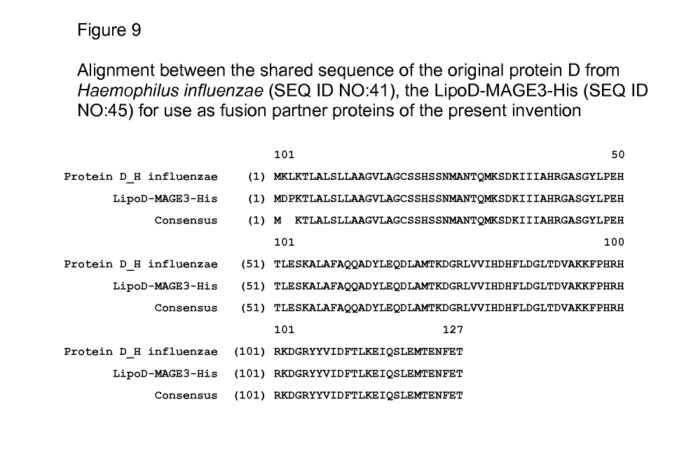

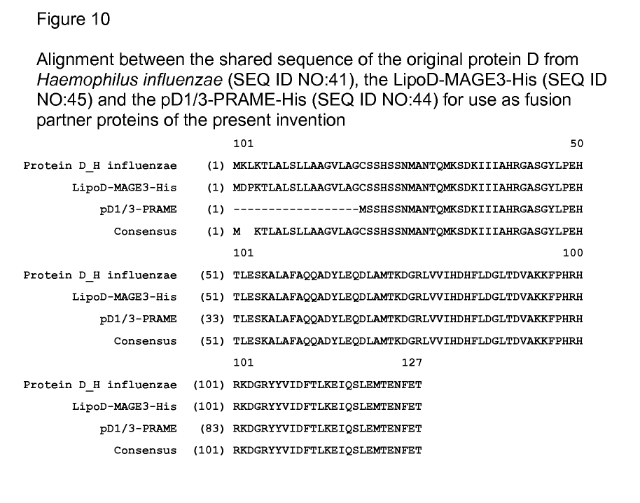

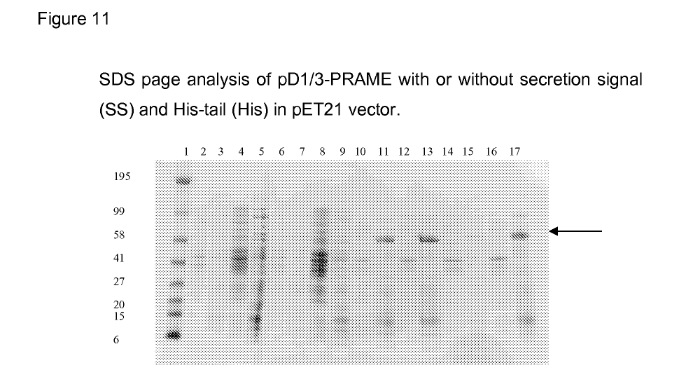

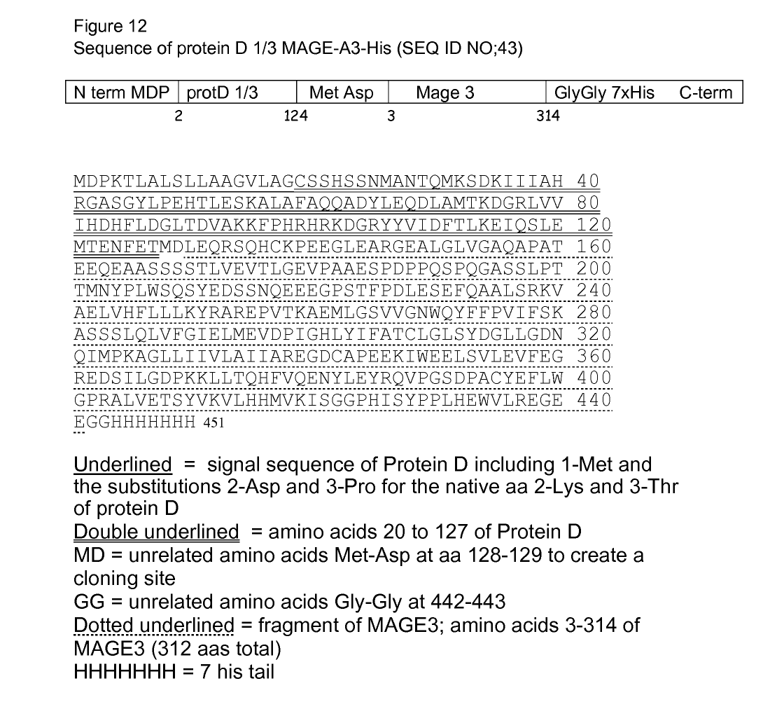

Brief Description of the Figures and Constructs/
Sequences

| | |
|---|---|
| FIG. 1 | SDS-page analysis of Construct 3 and |
| | 4 |
| FIG. 2 | SDS-page analysis of Construct 3a |
| | and 4a |
| FIG. 3 | CD4 response (AS01B adjuvant) |
| FIG. 4 | CD8 response (AS01B adjuvant- |
| | 20070499) |
| FIG. 5 | CD4 response (AS15 adjuvant) |
| FIG. 6 | CD8 response (AS15 adjuvant) |
| FIG. 7 | A marked up amino acid sequence of |
| | examples of constructs of the |
| | present invention |
| FIG. 8 | Alignment between LipoD-MAGE3-His |
| | (SEQ ID NO:43) and pDl/3-PRAME-His |
| | (SEQ ID NO:10) |
| FIG. 9 | Alignment between the shared |
| | sequence of the original protein D |
| | from Haemophilus influenzae (SEQ ID |
| | NO:45) and the LipoD-MAGE3-His (SEQ |
| | ID NO:43) |
| FIG. 10 | Alignment between the shared |
| | sequence of the original protein D |
| | from 1-Haemophilus influenzae (SEQ |
| | ID NO:41), the pD-MAGE3-His (SEQ ID |
| | NO:45) and a construct of pD1/3- |
| | PRAME-His which does not include the |
| | amino acids 2-D and 3-P (SEQ ID |
| | NO:44) |
| FIG. 11 | SDS page analysis of pD1/3-PRAME |
| | with or without secretion signal |
| | (SS) and His-tail (His) in pET21 |
| | vector. |
| FIG. 12 | Sequence of protein D 1/3 MAGE-A3- |
| | His (SEQ ID NO:44) |
| Construct 1 | MDP-20-127-Protein D-PRAME-TSGHHHHHH |
| (Example 1) | (plasmid TCMP14) (nucleotide |
| | sequence SEQ ID NO:1; amino acid |
| | sequence SEQ ID NO:2) |
| Construct 2 | MDP-20-127-Protein D-PRAME-no His |
| (Example 2) | tail (plasmid TCMP14) (nucleotide |
| | sequence SEQ ID NO:3; amino acid |
| | sequence SEQ ID NO:4) |
| Construct 3 | MDP-20-127-Protein D-PRAME-LEHHHHHH |
| (Example 3) | (plasmid pET21) (nucleotide sequence |
| | SEQ ID NO:5; amino acid sequence SEQ |
| | ID NO:6) |
| Construct 4 | MDP-20-127-Protein D-PRAME-no His |
| (Example 4) | tail (plasmid pET21) (nucleotide |
| | sequence SEQ ID NO:7; amino acid |
| | sequence SEQ ID NO:8) |
| Construct 3a | MDP-20-127-Protein D-PRAME-HHHHHH |
| (Example 3a) | (plasmid pET26) (nucleotide sequence |
| | SEQ ID NO:9; amino acid sequence SEQ |
| | ID NO:10) |
| Construct 4a | MDP-20-127-Protein D-PRAME-no His |
| (Example 4a) | tail (plasmid pET26) (nucleotide |
| | sequence SEQ ID NO:11; amino acid |
| | sequence SEQ ID NO:12) |
| Sequence 1 | is the DNA sequence for Example 1 |
| (SEQ ID NO:1) | |
| Sequence 2 | is the amino acid sequence for |
| (SEQ ID NO:2) | Example 1 |
| Sequence 3 | is the DNA sequence for Example 2 |
| (SEQ ID NO:3) | |
| Sequence 4 | is the amino acid sequence for |
| (SEQ ID NO:4) | Example 2 |
| Sequence 5 | is the DNA sequence for Example 3 |
| (SEQ ID NO:5) | |
| Sequence 6 | is the amino acid sequence for |
| (SEQ ID NO:6) | Example 3 |
| Sequence 7 | is the DNA sequence for Example 4 |
| (SEQ ID NO:7) | |
| Sequence 8 | is the amino acid sequence for |
| (SEQ ID NO:8) | Example 4 |
| Sequence 9 | is the codon optimized DNA sequence |
| (SEQ ID NO:9) | for Example 3a |
| Sequence 10 | is the amino acid sequence for |
| (SEQ ID NO:10) | Example 3a |
| Sequence 11 | is the codon optimized DNA sequence |
| (SEQ ID NO:11) | for Example 4a |
| Sequence 12 | is the amino acid sequence for |
| (SEQ ID NO:12) | Example 4a |
| SEQ ID NO:13 | VLDGLDVLL (PRA¹⁰⁰⁻¹⁰⁸) |
| SEQ ID NO:14 | SLYSFPEPEA (PRA¹⁴²⁻¹⁵¹) |
| SEQ ID NO:15 | ALYVDSLFFL (PRA³⁰⁰⁻³⁰⁹) |
| SEQ ID NO:16 | LYVDSLFFL (PRA³⁰¹⁻³⁰⁹) |
| SEQ ID NO:17 | SLLQHLIGL (PRA⁴²⁵⁻⁴³³) |
| SEQ ID NO:18 | Oligonucleotides used in the |
| to 35 | examples of the present invention |
| SEQ ID NO:36 | TCC ATG ACG TTC CTG ACG TT (CpG |
| | 1826;) |
| SEQ ID NO:37 | TCT CCC AGC GTG CGC CAT (CpG 1758) |
| SEQ ID NO:38 | ACC GAT GAC GTC GCC GGT GAC GGC ACC |
| | ACG TCG TCG TTT TGT CGT TTT GTC GTT |
| | (CpG 2006) |
| SEQ ID NO:39 | TCC ATG ACG TTC CTG ATG CT (CpG |
| | 1668) |
| SEQ ID NO:40 | TCG ACG TTT TCG GCG CGC GCC G (CpG |
| | 5456) |
| SEQ ID NO:41 | AA 1 to 127 of Protein D from |
| | Haemophilus influenzae |
| SEQ ID NO:42 | 1-Met; 2-Asp; 3-Pro; followed by AA |
| | 20 to 127 of Protein D |
| SEQ ID NO:43 | Amino acid sequence of protein D |
| | 1/3-MAGE-A3-His |
| SEQ ID NO:44 | Amino acids from protein D from |
| | Haemophilus influenzae for use in a |
| | pD1/3-PRAME-His sequence |
| SEQ ID NO:45 | Amino acids from protein D from |
| | Haemophilus influenzae for use in a |
| | pD1/3-MAGE-His sequence |

### SUMMARY OF THE INVENTION

The present invention provides a fusion protein comprising:
a) PRAME or an immunogenic fragment thereof, and
b) a heterologous fusion partner derived from protein D,
   wherein the said fusion protein does not include the secretion sequence (signal sequence) of protein D.

The present invention further provides a fusion partner protein as described herein derived from protein D, in which the fusion partner protein does not include the secretion sequence or signal sequence of protein D.

The present invention further provides a fusion protein as described herein and an antigen or fragment thereof.

The present invention further provides a fusion partner protein derived from protein D, in which the fusion partner protein comprises or consists of amino acids 20 to 127 of protein D. In one embodiment of the present invention, one or more amino acids from the protein D fusion partner protein as described herein may be deleted or may be replaced by substitution. The amino acids may be substituted with conservative substitutions as defined herein, or other amino acids may be used. In one embodiment, 1, 2, 3, 4, 5, 6, 7, 8, 9 or more amino acids may be substituted.

The protein D fusion partner protein as described herein may additionally or alternatively contain deletions or insertions within the amino acid sequence when compared to the wild-type protein D sequence. In one embodiment, 1, 2, 3, 4, 5, 6, 7, 8, 9 or more amino acids may be inserted or deleted.

The term “secretion sequence” or “signal sequence” or “secretion signal” of protein D, in the context of this application, is intended to refer to approximately amino acids 1 to 16, 17, 18 or 19 of the naturally occurring protein. In one embodiment, the secretion or signal sequence or secretion signal of protein D refers to the N-terminal 19 amino acids of protein D. The terms “secretion sequence” or “signal sequence” or “secretion signal” are used interchangeably in the present specification.

The fusion partner protein of the present invention may comprise the remaining full length protein D protein, or may comprise approximately the remaining N-terminal third of protein D. For example, the remaining N-terminal third of protein D may comprise approximately or about amino acids 20 to 127 of protein D. In one embodiment, the protein D sequence for use in the present invention comprises amino acids 20 to 127 of protein D. In a further embodiment, the present invention comprises or consists of any of the sequences starting from any of the following amino acids of the protein D sequence: 17, 18, 19, 20, 21, or 22; and terminating at any on the following amino acids of the protein D sequence: 125, 126, 127, 128, 129, 130, 131, 132, 133, 134, 135, 136, 137, 138, 139 or 140.

By “remaining” in this context is meant the sequence of the protein D protein without the secretion or signal sequence as described herein.

In one embodiment of the present invention in which the fusion protein comprises PRAME or an immunogenic fragment thereof, the protein D derivative of the present invention comprises approximately the first ⅓ of the protein, more specifically the amino acids 20 to 127. In an alternative embodiment of the present invention in which the fusion protein comprises PRAME or an immunogenic fragment thereof, the protein D comprises approximately the first ⅓ of the protein in which the N-termninal 109 amino acids of protein D are used. In one embodiment of the present invention the protein D portion does not include the secretion sequence of the protein. In one embodiment of the present invention the protein D derivative is not lipidated.

In one embodiment, the present invention provides a protein D construct, as described herein, as a fusion partner protein. The protein D construct may be a fusion partner protein for a construct additionally comprising a PRAME or MAGE-A3 construct as described herein or may be a fusion partner protein for a construct additionally comprising another cancer antigen or any other antigen.

It seems that for fusion proteins comprising PRAME or an immunogenic fragment thereof and protein D, or for fusion proteins comprising protein D, or for a fusion partner protein comprising protein D, that the presence of the secretion sequence (or signal sequence) may detrimentally affect the amount of fusion protein produced.

### PRAME

In one aspect the fusion protein of the present invention comprises a fusion partner protein as described herein and a PRAME antigen or immunogenic fragment thereof. Generally the PRAME protein has 509 amino acids and in one embodiment all 509 amino acids of PRAME may be used. Several cytotoxic T lymphocytle (CTL) epitopes have been identified on PRAME, for example:

| | |
|---|---|
| VLDGLDVLL; | (PRA¹⁰⁰⁻¹⁰⁸; SEQ ID NO:13) |
| SLYSFPEPEA; | (PRA¹⁴²⁻¹⁵¹; SEQ ID NO:14) |
| ALYVDSLFFL; | (PRA³⁰⁰⁻³⁰⁹; SEQ ID NO:15) |
| LYVDSLFFL | (PRA³⁰¹⁻³⁰⁹; SEQ ID NO:16) |
| and | |
| SLLQHLIGL. | (PRA⁴²⁵⁻⁴³³; SEQ ID NO:17) |

Generally it is desirable to include as many of these epitopes as possible into the antigen to generate a strong immune response and ensure the antigen is as immunogenic as possible. Although, it may be possible to compensate for a lower immunogenicity of a given construct by employing a formulation with a potent immunological adjuvant. Strong adjuvants are discussed below in more detail.

In one aspect the invention provides the PRAME portion of the fusion protein comprising, consisting of or consisting essentially of full length protein.

However, the invention also extends to PRAME constructs with conservative substitutions. In one embodiment, 1, 2, 3, 4, 5, 6, 7, 8, 9 or more amino acids may be substituted. The PRAME construct as described herein may additionally or alternatively contain deletions or insertions within the amino acid sequence when compared to the wild-type PRAME sequence. In one embodiment, 1, 2, 3, 4, 5, 6, 7, 8, 9 or more amino acids may be inserted or deleted.

Conservative substitutions are well known and are generally set up as the default scoring matrices in sequence alignment computer programs. These programs include PAM250 (Dayhoft M. O. et al., (1978), “A model of evolutionary changes in proteins”, In “Atlas of Protein sequence and structure” 5(3) M. O. Dayhoft (ed.), 345-352), National Biomedical Research Foundation, Washington, and Blosum 62 (Steven Henikoft and Jorja G. Henikoft (1992), “Amino acid substitution matricies from protein blocks”), Proc. Natl. Acad. Sci. USA 89 (Biochemistry): 10915-10919.

In general terms, substitution within the following groups are conservative substitutions, but substitutions between groups are considered non-conserved. The groups are:
i) Aspartate/asparagine/glutamate/glutamine
ii) Serine/threonine
iii) Lysine/arginine
iv) Phenylalanine/tyrosine/tryptophane
v) Leucine/isoleucine/valine/methionine
vi) Glycine/alanine

Generally the PRAME sequence/amino acids used in the fusion proteins of the invention will be greater than 80%, such as 85, 90, 95 and more specifically 99% identical to naturally occurring PRAME. However, those skilled in the art are aware that amino acid residues generated as a result of the cloning process may be retained in the recombinantly synthesized proteins. If these do not detrimentally affect the characteristics of the product, it is optional whether or not they are removed.

In one aspect the invention provides a fusion protein as described herein comprising, consisting of or consisting essentially of full length PRAME protein. In a further aspect the PRAME portion of the fusion protein of the present invention comprises, consists of or consists essentially of one or more of the following epitopes:

| | |
|---|---|
| VLDGLDVLL; | (PRA¹⁰⁰⁻¹⁰⁸; SEQ ID NO:13) |
| SLYSFPEPEA; | (PRA¹⁴²⁻¹⁵¹; SEQ ID NO:14) |
| ALYVDSLFFL; | (PRA³⁰⁰⁻³⁰⁹; SEQ ID NO:15) |
| LYVDSLFFL | (PRA³⁰¹⁻³⁰⁹; SEQ ID NO:16) |
| and | |
| SLLQHLIGL. | (PRA⁴²⁵⁻⁴³³; SEQ ID NO:17) |

### Fusion Proteins

In a further embodiment of the present invention, a tumour antigen other than PRAME or in addition to PRAME may be used in a fusion protein as described herein. In one embodiment, a fusion protein is provided comprising a fusion partner protein as described herein and one or more of the following tumour antigens or tumour antigen derivatives or an immunogenic portion thereof which is able to direct an immune response to the antigen: a MAGE antigen, for example a MAGE-A antigen such as MAGE 1, MAGE 2, MAGE 3, MAGE 4, MAGE 5, MAGE 6, MAGE 7, MAGE 8, MAGE 9, MAGE 10, MAGE 11, MAGE 12. These antigens are sometimes known as MAGE A1, MAGE A2, MAGE A3, MAGE A4, MAGE A5, MAGE A6, MAGE A7, MAGE A8, MAGE A9, MAGE A 10, MAGE A11 and/or MAGE A12 (The MAGE A family). In one embodiment, an antigen from one of two further MAGE families may be used: the MAGE B and MAGE C group. The MAGE B family includes MAGE B1 (also known as MAGE Xp1, and DAM 10), MAGE B2 (also known as MAGE Xp2 and DAM 6) MAGE B3 and MAGE B4—the Mage C family currently includes MAGE C1 and MAGE C2.

The MAGE antigen for use in the present invention may comprise the full length MAGE antigen. Alternatively, the MAGE antigen may comprise an immunogenic portion of MAGE in which 1, 2, 3, 4, 5, 6, 7, 8, 9, 10 or more amino acids may be deleted from or substituted in the amino acid sequence. In one embodiment of the present invention, 2 amino acids may be deleted from the N-terminus of the MAGE sequence. In one embodiment of the present invention in which the antigen is MAGE-A3 or an immunogenic portion thereof, the sequence of MAGE-A3 may be from amino acid 3 to 314 of MAGE-A3.

In another embodiment, the tumour antigen or derivative for use in the present invention may be PRAME, BAGE, LAGE 1, LAGE 2 (also known as NY-ESO-1), SAGE, HAGE, XAGE, PSA, PAP, PSCA, P501S (also known as prostein), HASH1, HASH2, Cripto, B726, NY-BR1.1, P510, MUC-1, Prostase, STEAP, tyrosinase, telomerase, survivin, CASB616, P53, and/or Her-2/neu or an immunogenic portion thereof which is able to direct an immune response to the antigen.

In a further embodiment of the invention, the tumour antigen may comprise or consist of one of the following antigens, or an immunogenic portion thereof which is able to direct an immune response to the antigen: SSX-2; SSX-4; SSX-5; NA17; MELAN-A; P790; P835; B305D; B854; CASB618 (as described in WO00/53748); CASB7439 (as described in WO01/62778); C1491; C1584; and C1585.

In one embodiment, the antigen for use in the present invention may comprise or consist of P501S. P501S, also named prostein (Xu et al., Cancer Res. 61, 2001, 1563-1568), is known as SEQ ID NO. 113 of WO98/37814 and is a 553 amino acid protein. Immunogenic fragments and portions thereof comprising at least 20, preferably 50, more preferably 100 contiguous amino acids as disclosed in the above referenced patent application may be used in fusion proteins of the present invention. Preferred fragments are disclosed in WO 98/50567 (PS108 antigen) and as prostate cancer-associated protein (SEQ ID NO: 9 of WO 99/67384). Other preferred fragments are amino acids 51-553, 34-553 or 55-553 of the full-length P501S protein.

In one embodiment, the antigen may comprise or consist of WT-1 expressed by the Wilm's tumor gene; or an immunogenic portion thereof which is able to direct an immune response to the antigen; or the N-terminal fragment WT-1F comprising about or approximately amino acids 1-249 of WT-1.

In a further embodiment, the antigen may comprise or consist of the antigen expressed by the Her-2/neu gene, or a fragment thereof or an immunogenic portion thereof which is able to direct an immune response to the antigen. In one embodiment, the Her-2/neu antigen may be one of the following fusion proteins which are described in WO00/44899.

The antigen for use in the present invention may comprise or consist of “HER-2/neu ECD-ICD fusion protein,” also referred to as “ECD-ICD” or “ECD-ICD fusion protein,” which refers to a fusion protein (or fragments thereof) comprising the extracellular domain (or fragments thereof) and the intracellular domain (or fragments thereof) of the HER-2/neu protein. In one embodiment, this ECD-ICD fusion protein does not include a substantial portion of the HER-2/neu transmembrane domain, or does not include any of the HER-2/neu transmembrane domain.

In a further embodiment, the antigen may comprise or consist of “HER-2/neu ECD-PD fusion protein,” also referred to as “ECD-PD” or “ECD-PD fusion protein,” or the “HER-2/neu ECD-ΔPD fusion protein,” also referred to as “ECD-ΔPD” or “ECD-ΔPD fusion protein,” which refers to fusion proteins (or fragments thereof) comprising the extracellular domain (or fragments thereof) and phosphorylation domain (or fragments thereof, e.g., ΔPD) of the HER-2/neu protein. In one embodiment, the ECD-PD and ECD-ΔPD fusion proteins do not include a substantial portion of the HER-2/neu transmembrane domain, or does not include any of the HER-2/neu transmembrane domain.

The fusion proteins of the PRAME antigen and protein D fusion partner protein as described herein may be chemically conjugated, but are preferably expressed as recombinant fusion proteins, which may allow increased levels of PRAME protein to be produced in an expression system as compared to PRAME alone without fusion partner, such as protein D or modified protein D proteins.

Additionally or alternatively, the tumour antigens described herein and the fusion partner protein of the present invention may be chemically conjugated or may be expressed as recombinant fusion proteins, which may allow increased levels of PRAME protein or another tumour antigen to be produced in an expression system as compared to PRAME or another tumour antigen alone without fusion partner, such as protein D or modified protein D proteins.

Fusion proteins of the present invention, as described herein, may additionally comprise one or more linker sequences between the fusion partner protein and the tumour antigen or immunogenic portion thereof, or between the fusion partner protein and a His tail or other affinity tag (if present); or between the tumour antigen or immunogenic portion thereof and a His tail or other affinity tag (if present). The amino acids in the linker sequences may be unrelated to the sequences of the antigen and/or fusion partner.

Fusion proteins of the present invention, as described herein, may additionally comprise amino acids Met-Asp-Pro at the N-terminal end of the fusion protein sequence. The Met amino acid may be from the original protein D sequence or may be from an unrelated sequence.

The fusion partner may assist in expressing the protein (expression enhancer) at higher yields than the native recombinant protein. The fusion partner protein D, due to its foreign nature, may be particularly immunogenic in vivo and assist the fusion protein comprising PRAME or another tumour antigen by providing T helper epitopes, preferably T helper epitopes recognized by CD4 T-cells. Such CD4-T cells may be believed to contribute to generating a favourable immune response, in particular, a CD8 cytolytic T-cell response.

In one embodiment, the fusion partner may act as both an expression enhancing partner and an immunological fusion partner.

In one aspect the invention provides a fusion protein wherein the N-terminal portion of protein D (as described above or herein) is fused to the N-terminus of PRAME or an immunogenic fragment thereof. More specifically the fusion with the protein D fragment and the N-terminus of PRAME is effected such that the PRAME replaces the C-terminal-fragment of protein D that has been excised. Thus the N-terminus of protein D becomes the N-terminus of the fusion protein.

In a further aspect the invention provides a fusion protein wherein the N-terminal portion of protein D (as described above or herein) is fused to the N-terminus or another portion of a tumour antigen or an immunogenic fragment thereof. More specifically the fusion with the protein D fragment and the N-terminus or other portion of a tumour antigen may be effected such that the PRAME or other tumour antigen or derivative thereof as described herein replaces the C-terminal-fragment of protein D that has been excised. Thus the N-terminus of protein D becomes the N-terminus of the fusion protein.

Other fusion partners or fragments thereof may be included in fusion proteins of the invention or may replace the protein D element of the present invention, for example in embodiments comprising the PRAME antigen or a fragment or portion thereof as described herein. Examples of other fusion partners include:
the non-structural protein from influenzae virus, NS1 (hemagglutinin)—typically the N terminal 81 amino acids are utilized, although different fragments may be used provided they include T-helper epitopes,
LYTA derived from Streptococcus pneumoniae, which synthesize an N-acetyl-L-alanine amidase, amidase LYTA, (coded by the lytA gene {Gene, 43 (1986) page 265-272} such as the repeat portion of the Lyta molecule found in the C terminal end for example starting at residue 178 such as residues 188-305.

Purification of hybrid proteins containing the C-LYTA fragment at its amino terminus has been described {Biotechnology: 10, (1992) page 795-798.

Fusion proteins of the invention may include an affinity tag, such as for example, a histidine tail comprising between 5 to 9 such as 6 histidine residues. These residues may, for example be on the terminal portion of protein D (such as the N-terminal of protein D) and/or the may be fused to the terminal portion of the PRAME antigen or derivative thereof, or the tumour antigen or derivative thereof as described herein. Generally however the histidine tail with be located on terminal portion of the PRAME antigen or derivative thereof, or the tumour antigen or derivative thereof as described herein such as the C-terminal end of the PRAME antigen or derivative thereof, or the tumour antigen or derivative thereof as described herein. Histidine tails may be advantageous in aiding purification.

The present invention also provides a nucleic acid encoding the proteins of the present invention. Such sequences can be inserted into a suitable expression vector and used for DNA/RNA vaccination or expressed in a suitable host. Microbial vectors expressing the nucleic acid may be used as vaccines. Such vectors include for example, poxvirus, adenovirus, alphavirus, listeria and monophage.

A DNA sequence encoding the proteins of the present invention can be synthesized using standard DNA synthesis techniques, such as by enzymatic ligation as described by D. M. Roberts et al. in Biochemistry 1985, 24, 5090-5098, by chemical synthesis, by in vitro enzymatic polymerization, or by PCR technology utilizing for example a heat stable polymerase, or by a combination of these techniques.

Enzymatic polymerization of DNA may be carried out in vitro using a DNA polymerase such as DNA polymerase I (Klenow fragment) in an appropriate buffer containing the nucleoside triphosphates dATP, dCTP, dGTP and dTTP as required at a temperature of 10°-37° C., generally in a volume of 50 μl or less. Enzymatic ligation of DNA fragments may be carried out using a DNA ligase such as T4 DNA ligase in an appropriate buffer, such as 0.05M Tris (pH 7.4), 0.01M MgCl₂, 0.01M dithiothreitol, 1 mM spermidine, 1 mM ATP and 0.1 mg/ml bovine serum albumin, at a temperature of 4° C. to ambient, generally in a volume of 50 ml or less. The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982), or in other scientific publications, for example M. J. Gait, H. W. D. Matthes, M. Singh, B. S. Sproat, and R. C. Titmas, Nucleic Acids Research, 1982, 10, 6243; B. S. Sproat, and W. Bannwarth, Tetrahedron Letters, 1983, 24, 5771; M. D. Matteucci and M. H. Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biernat, J. McMannus, and H. Koester, Nucleic Acids Research, 1984, 12, 4539; and H. W. D. Matthes et al., EMBO Journal, 1984, 3, 801.

The process of the invention may be performed by conventional recombinant techniques such as described in Maniatis et al., Molecular Cloning—A Laboratory Manual; Cold Spring Harbor, 1982-1989.

In particular, the process may comprise the steps of:
i) preparing a replicable or integrating expression vector capable, in a host cell, of expressing a DNA polymer comprising a nucleotide sequence that encodes the protein or an immunogenic derivative thereof;
ii) transforming a host cell with said vector;
iii) culturing said transformed host cell under conditions permitting expression of said DNA polymer to produce said protein; and
iv) recovering said protein.

The term ‘transforming’ is used herein to mean the introduction of foreign DNA into a host cell. This can be achieved for example by transformation, transfection or infection with an appropriate plasmid or viral vector using e.g. conventional techniques as described in Genetic Engineering; Eds. S. M. Kingsman and A. J. Kingsman; Blackwell Scientific Publications; Oxford, England, 1988. The term ‘transformed’ or ‘transformant’ will hereafter apply to the resulting host cell containing and expressing the foreign gene of interest.

The expression vectors are novel and also form part of the invention.

The replicable expression vectors may be prepared in accordance with the invention, by cleaving a vector compatible with the host cell to provide a linear DNA segment having an intact replicon, and combining said linear segment with one or more DNA molecules which, together with said linear segment encode the desired product, such as the DNA polymer encoding the protein of the invention, or derivative thereof, under ligating conditions.

Thus, the DNA polymer may be preformed or formed during the construction of the vector, as desired.

The choice of vector will be determined in part by the host cell, which may be prokaryotic or eukaryotic but are generally E. coli or CHO cells. Suitable vectors may include plasmids for example TMCP14 or pET21 or pET26, pcDNA3, bacteriophages, cosmids and recombinant viruses.

The preparation of the replicable expression vector may be carried out conventionally with appropriate enzymes for restriction, polymerization and ligation of the DNA, by procedures described in, for example, Maniatis et al. cited above.

The recombinant host cell is prepared, in accordance with the invention, by transforming a host cell with a replicable expression vector of the invention under transforming conditions. Suitable transforming conditions are conventional and are described in, for example, Maniatis et al. cited above, or “DNA Cloning” Vol. II, D. M. Glover ed., IRL Press Ltd, 1985.

The choice of transforming conditions is determined by the host cell. Thus, a bacterial host such as E. coli may be treated with a solution of CaCl₂ (Cohen et al., Proc. Nat. Acad. Sci., 1973, 69, 2110) or with a solution comprising a mixture of RbCl, MnCl₂, potassium acetate and glycerol, and then with 3-[N-morpholino]-propane-sulphonic acid, RbCl and glycerol. Mammalian cells in culture may be transformed by calcium co-precipitation of the vector DNA onto the cells. The invention also extends to a host cell transformed with a replicable expression vector of the invention.

The DNA may be codon optimized by standard techniques to further facilitate expression of the relevant host. In one embodiment of the present invention there is provided DNA encoding a fusion protein comprising a PRAME antigen or portion or fragment thereof as described herein, in which the nucleotide sequence of the PRAME antigen or portion or fragment thereof is codon-optimized. In one embodiment, the protein D nucleotide sequence is not codon-optimized.

Culturing the transformed host cell under conditions permitting expression of the DNA polymer is carried out conventionally, as described in, for example, Maniatis et al. and “DNA Cloning” cited above. Thus, preferably the cell is supplied with nutrient and cultured at a temperature below 50° C.

The proteins of the present invention may be expressed in prokaryotes or eukaryotes such as yeast but are often expressed in E. Coli. Particular strains of E. coli such as AR58 and BLR DE3 may be employed.

Generally a selection marker of, for example kanamycine resistance or ampicillin resistance is incorporated to facilitate identification of the successful incorporation of the recombinant gene/construct into the expression system.

The product is recovered by conventional methods according to the host cell and according to the localization of the expression product (intracellular or secreted into the culture medium or into the cell periplasm). In one embodiment of the present invention the expression product is intracellular. In one embodiment of the present invention the expression product is an insoluble protein. Thus, where the host cell is bacterial, such as E. coli it may, for example, be lysed physically, chemically or enzymatically and the protein product isolated from the resulting lysate. Where the host cell is mammalian, the product may generally be isolated from the nutrient medium or from cell free extracts. Conventional protein isolation techniques include selective precipitation, adsorption chromatography, and affinity chromatography including a monoclonal antibody affinity column.

In one embodiment of the invention there is provided a process for producing a fusion protein as described herein comprising the step of expressing in a cell a fusion protein comprising a fusion partner protein as described herein. The cell may be a bacterium. In one embodiment in which the cell is a bacterium, the bacterium may be E. coli. The process of the present invention may comprise the step of expressing a fusion protein as described herein in a cell as an insoluble protein. The process may further comprise the step of lysing the cell and purifying the expressed fusion protein from the lysed cells.

In one embodiment of the invention there is provided a fusion protein obtained by or obtainable by a method or process described herein.

The proteins of the present invention are provided either soluble in a liquid form or in a lyophilized form.

It is generally expected that each human dose will comprise 1 to 1000 μg of protein, and preferably 30-300 μg.

The present invention also provides pharmaceutical composition such as vaccine comprising a fusion protein of the present invention in a pharmaceutically acceptable excipient.

The vaccine may optionally contain one or more other tumour-associated antigens or polypeptides, or preferably be combined with other cancer vaccines based on a tumour-associated antigen. For example, these tumour-associated antigens could be antigens as described herein and/or may be members belonging to the MAGE, LAGE and GAGE families or WT-1. In one embodiment the tumour-associated antigen may comprise or consist of the MAGE-A3 antigen.

Vaccine preparation is generally described in Vaccine Design (“The subunit and adjuvant approach” (eds. Powell M. F. & Newman M. J). (1995) Plenum Press New York). Encapsulation within liposomes is described by Fullerton, U.S. Pat. No. 4,235,877.

The proteins of the present invention may be preferably adjuvanted in the vaccine formulation of the invention. Suitable adjuvants may include an aluminum salt such as aluminum hydroxide gel (alum) or aluminum phosphate, but may also be a salt of calcium, iron or zinc, or may be an insoluble suspension of acylated tyrosine, or acylated sugars, cationically or anionically derivatized polysaccharides, or polyphosphazenes. Other known adjuvants include CpG containing oligonucleotides. The oligonucleotides are characterized in that the CpG dinucleotide is unmethylated. Such oligonucleotides are well known and are described in, for example WO 96/02555.

In the formulation of the inventions it may be desirable that the adjuvant composition induces an immune response preferentially of the TH1 type. In one embodiment there is provided an adjuvant system including, for example a combination of monophosphoryl lipid A, preferably 3-de-O-acylated monophosphoryl lipid A (3D-MPL) together with an aluminum salt. The adjuvant may optionally also include CpG oligonucleotides to preferentially induce a TH1 response.

An enhanced system that may be used in the present invention comprises the combination of a monophosphoryl lipid A and a saponin derivative particularly the combination of QS21 and 3D-MPL as disclosed in WO 94/00153, or, for example a less reactogenic composition where the QS21 is quenched with cholesterol as disclosed in WO 96/33739.

A formulation that may be used in formulations of the present invention, comprising QS21 3D-MPL & tocopherol, for example in an oil in water emulsion, is described in WO 95/17210.

Another adjuvant formulation that may be used in formulations of the present invention is QS21, 3D-MPL & CpG or equivalent thereof, for example in an oil in water emulsion or as a liposomal formulation.

Accordingly in one embodiment of the present invention there is provided a vaccine comprising a fusion protein or fusion partner protein as described herein and an adjuvant, for example as described above.

### Combination of PRAME and MAGE

In one embodiment of the present invention there is provided a composition comprising (a) an antigen component comprising a PRAME antigen or fusion protein as described herein and (b) an antigen component comprising a MAGE antigen or fusion protein as described herein. In one embodiment, the composition may further comprise an adjuvant as described herein.

The MAGE antigen for use in the combination may comprise the full length MAGE antigen. Alternatively, the MAGE antigen may comprise an immunogenic portion of MAGE in which 1, 2, 3, 4, 5, 6, 7, 8, 9, 10 or more amino acids may be deleted from or substituted in the amino acid sequence. In one embodiment of the present invention, 2 amino acids may be deleted from the N-terminus of the MAGE sequence. In one embodiment of the present invention in which the antigen is MAGE-A3 or an immunogenic portion thereof, the sequence of MAGE-A3 may be from amino acid 3 to 314 of MAGE-A3.

For the combination described above, either or both of the PRAME and/or MAGE antigens may be part of a fusion protein or proteins as described herein, or the antigens may be present in other fusion proteins or may be presented as antigen alone.

In one embodiment of the present invention there is provided a composition comprising a fusion protein comprising a PRAME antigen and fusion partner protein as described herein and a fusion protein comprising a MAGE-A3 antigen and fusion partner protein as described herein. In an alternative embodiment, the fusion protein comprising the MAGE-A3 antigen comprises or consists of the MAGE-A3 antigen and a fusion partner protein comprising approximately the first 109 amino acids of protein D, in which one or two or more amino acids from protein D are optionally substituted, and in which the signal sequence of protein D is optionally present, in addition to the first 109 amino acids of protein D.

The fusion proteins of the present invention may additionally optionally comprise one or more amino acids as “linker” between the sequences of the antigen and the fusion partner protein or between the antigen and a His tail, if present. The amino acids may be unrelated to the sequences of the antigen and/or fusion partner.

Fusion proteins of the present invention, as described herein, may additionally comprise amino acids Met-Asp-Pro at the N-terminal end of the fusion protein sequence. The Met amino acid may be from the original protein D sequence or may be from an unrelated sequence.

In one embodiment, the sequence of a fusion protein comprising MAGE-A3 and protein D for use in the present invention is shown in FIG. 12 and SEQ ID NO:43.

The present invention also extends to methods of preparing said vaccines/compositions.

### EXAMPLES

Four fusion constructs were prepared and will be referred to herein as Examples/constructs 1, 2, 3 and 4. A codon optimized construct was prepared from example 3 and is designated as example 3a herein. A codon optimized construct was prepared from example 4 and is designated as example 4a herein.

In Examples 3a and 4a the sequence in respect of the protein D portion of the molecule is the same. However, certain codons in the PRAME region were modified, to further improve expression and, in Example 3a, the linker between PRAME and the his tail has been removed.

TABLE A
Fusion Protein Structures and Plasmid Details of the Examples/Constructs
1 to 4

| | N-terminal -------------------------------------------------- C-Terminal | | | | |
|---|---|---|---|---|---|
| | amino acids | | | | |
| Example/ | inserted by | fusion | | Linker/His tail | plasmid |
| Construct No. | cloning process | partner | cancer antigen | amino acid seq. | used |
| 1 | MDP | 20-127 | PRAME | TSGHHHHHH | TCMP14 |
| | | protein D | | | |
| 2 | MDP | 20-127 | PRAME | — | TCMP14 |
| | | protein D | | | |
| 3 | MDP | 20-127 | PRAME | LEHHHHHH | pET21 |
| | | protein D | | | |
| 4 | MDP | 20-127 | PRAME | — | pET21 |
| | | protein D | | | |
| 3a | MDP | 20-127 | PRAME | HHHHHH | pET26 |
| | | protein D | | | |
| 4a | MDP | 20-127 | PRAME | — | pET26 |
| | | protein D | | | |

The fusion proteins of the above examples comprise the amino acids 20-127 of protein D. The amino acids Met, Asp and Pro were included at the N-terminal of the protein D fragment (ie amino acids MDP-20-127 Protein D). It is thought that these three additional amino acids may aid the stability of the protein and/or increase the level of the protein expression thereof. Amino acid 127 of protein D is fused to the N-terminal of full length PRAME (ie amino acid 127 of protein D is fused to N-terminal of PRAME). A histidine tag tail, to aid purification, was included in three of the six proteins. The exact sequence of the tail is dependent the plasmid used.

Three different types of plasmids, TCMP14 and pET21 or pET26 were constructed: for each plasmid, DNA encoding for fusion protein was included with and without a histidine tail.

Unless stated otherwise the general strategy below was used in the preparation of each of the examples.

### Cloning Strategy for the Generation of PD⅓-PRAME (With or Without His-Tag) Recombinant Protein Using TCMP14 Vector:

Amplification of the sequences presented in the plasmid TCMP14 were done using a three steps PCR strategy. The vector pHIC348 containing the DNA sequence encoding the entire protein D gene has been obtained from Dr. A. Forsgren, Department of Medical Microbiology, University of Lund, Malmö General Hospital, Malmö, Sweden. The DNA sequence of protein D has been published by Janson et al. (1991) {Janson H, L O Heden, A Grubb, M Ruan, & A Forsgren. 1991. Infect Immun 59:119-125}. The expression vector pMG81 is a derivative of pBR322, in which bacteriophage λ derived control elements for transcription and translation of foreign inserted genes were introduced (Shatzman et al., 1983) {Shatzman A, Y S Ho, & M Rosenberg. 1983. Experimental Manipulation of Gene Expression. Inouya (ed) pp 1-14. Academic NY}. In addition, the Ampicillin resistance gene was exchanged with the Kanamycin resistance gene. The coding sequence for the portion of NS1 protein (amino acid 4 to 81) was substituted for a multiple cloning sites to get pMG81 MCS. The coding sequence for the ⅓ protein D (amino acid 20 to 127) was cloned into pMG81 MCS using BamHI and NcoI restriction sites to get pMG81-⅓PD. First, PCR amplification of the section corresponding to amino acid 20-127 of protein D was done using pMG81-⅓PD vector as template and oligonucleotide sense:

5′ ATA TAA CAT ATG GAT CCA AGC AGC CAT TCA TCA
AAT 3′ (CAN008; SEQ ID NO:18)

and antisense:

5′ CCA CAA ACG CCT TCG TTC CAT GGT TTC AAA GTT TTC
TGT C 3′ (CAN037; SEQ ID NO: 19).

PRAME cDNA obtained from the Ludwig Institute, Brussels, Belgium was inserted in the Bstx1-Not1 sites of the pCDNA1 vector (Invitrogen) to generate pCDNA-1-PRAME recombinant vector. PCR amplification of the section corresponding to amino acid of PRAME protein was done using pcDNA-1-PRAME vector (GSKBio) as template and oligonucleotide sense:

5′ GAC AGA AAA CTT TGA AAC CAT GGA ACG AAG GCG TTT
GTG G 3′ (CAN036; SEQ ID NO: 20)

and antisense:

5′ AGA GAG ACT AGT CTA GTT AGG CAT GAA ACA GGG GCA
CAG 3′ (CAN029; SEQ ID NO: 21)
or
5′ GGA GGA ACT AGT GTT AGG CAT GAA ACA GGG GCA
CAG 3′ (CAN002; SEQ ID NO: 22)

depending if a his-tail (CAN002) or not (CAN029) was added. The final PRAME sequence inserted in the TCMP14 plasmid was obtained following a PCR amplifications using the ⅓PD and PRAME gene templates that were generated in the preliminary steps for template and oligonucleotide sense: CAN008, and antisense: CAN029 or CAN002 depending if an his-tail was present (CAN002) or not (CAN029). NdeI at 5′ end and SpeI at 3′ end sites were also added for cloning of the fragment into TCMP14 vector. Construction of the vector design to express the recombinant protein ⅓PD-PRAME with or without His-tag recombinant protein using pET21 vector:

A recombinant cDNA plasmid called pcDNA1-PRAME (as described in the previous strategy) containing the coding sequence for PRAME gene and the vector PMG81-⅓PD (as described in the previous strategy) containing the N-terminal portion of the protein D coding sequence were used. The cloning strategy included the following steps.
a) First, the ⅓PD sequence without secretion signal (secretion or signal sequence) was PCR amplified from plasmid PMG81-⅓PD using the oligonucleotide sense:

5′ AGAGAGCATATGAGCAGCCATTCATCAAATATGGCG
(CAN04; SEQ ID NO: 22),

and the antisense:

5′ ACGTGGGCGGCCGCGGTTTCAAAGTTTTCTGTCATTTCTAA
(CAN032; SEQ ID NO: 23);

Nde1 at the 5′ end and Not1 at the 3′ end sites were also added for cloning of the fragment into pET21b(+) vector.
b) The PRAME sequence was PCR amplified from plasmid pcDNA1-PRAME using the oligonucleotide sense:

5′ TTGTTGGCGGCCGCAATGGAACGAAGGCGTTTGTGGGGT
(CAN033; SEQ ID NO: 25),

and the antisense:

5′ GGAGGACTCGAGGTTAGGCATGAAACAGGGGCACAG
(CAN034; SEQ ID NO: 26);

Not1 at the 5′ end and Xho1 at the 3′ end sites were also added for cloning of the fragment into pET21b vector. This amplification resulted in the addition at the C-terminal of the protein of two amino acids, Leu and Glu, followed by 6 His in pET21b(+) plasmid. For the generation of the protein without His-tag, a stop codon (TAG) was added at the 3′ end of the PRAME gene by using CAN033 and CAN035 (antisense:

5′ GGAGGACTCGAGCTAGTTAGGCATGAAACAGGGGCACAG
(CAN035; SEQ ID NO: XX)

instead of CAN033 and CAN034.
c) Cloning into pET21b(+) plasmid (Invitrogen) of the above amplified fragments.
d) Removal of Not1 site between ⅓PD and PRAME by using QuikChange II Site-Directed Mutagenesis Kit (Stratagene) and the oligonucleotide sense:

5′ CAGAAAACTTTGAAACCATGGAACGAAGGCG
(CAN106; SEQ ID NO: XX),

and the antisense:

5′ cgccttcgttccatggtttcaaagttttctg
(CAN107; SEQ ID NO: XX).

e) Addition of two amino acids Asp and Pro following the Met at position 1 at the N-terminal of the protein D ⅓ by mutagenesis and using the oligonucleotide sense:

5′ GGAGATATACATATGGATCCAAGCAGCCATTCATCAAATATGG
(CAN104; SEQ ID NO: XX)

and the antisense:

5′ CCATATTTGATGAATGGCTGCTTGGATCCATATGTATATCTCC
(CAN105; SEQ ID NO: XX).

Construction of the Vector Design to Express the Recombinant Protein ⅓PD-PRAME Codon Optimized (Without or With His Tag) in pET26 Vector:

The PRAME gene was codon optimized and cloned in pGA4 backbone with the addition of Not1 and Xho1 sites in the 5′ end and the 3′ end of the optimized gene respectively.

This plasmid, named 0606420pGA4, was used to clone the gene in fusion with the PD⅓ in the pET26 vector using the following steps.
a) Removal of the Not1/Xho1 fragment corresponding to the optimized PRAME sequence with a stop codon at the 3′ end of the gene from 0606420pGA4 plasmid.
b) Cloning of the optimized PRAME fragment into a pET26b(+) plasmid which contain the ⅓PD previously cloned Nde1/Not1 with CAN040 and CAN032 oligonucleotides as described above and where Asp and Pro amino acids were added in N-terminal by mutagenesis method with CAN104 and CAN105 oligonucleotides.
c) Removal of the Not1 site by mutagenesis with oligonucleotides: sense

5′ GACAGAAAACTTTGAAACCATGGAACGTCGTCGTCTGTGG
(CAN123; SEQ ID NO: XX)

and antisense

5′ CCACAGACGACGACGTTCCATGGTTTCAAAGTTTTCTGTC
(CAN124; SEQ ID NO: XX).

This resulted in ⅓PD-PRAME codon optimized fusion protein without His-tail.
d) The plasmid was then used as a template for the generation of ⅓PD-PRAME codon optimized with 6 His plasmid. PCR amplification of the fusion protein was done with oligonucleotides sense

5′ GGAATTCCATATGGATCCAAGCAGCCATTC
(CAN199; SEQ ID NO: XX)

and a antisense

5′ GGAGCTCTCGAGTCAGTGGTGGTGGTGGTGGTGGTTCGGCATAAAGC
ACGGGC (CAN 198; SEQ ID NO: XX);

Nde1 at the 5′ end, Xho1 at the 3′ end sites followed by 6 His and a stop codon were also added for cloning of the fragment into pET26b(+) vector.
e) Cloning of the amplified fragment in pET26b(+) plasmid from Invitrogen.

For the production of the fusion protein, the DNA construct has been cloned into the expression vector TCMP14. This plasmid utilizes signals from lambda phage DNA to drive the transcription and translation of inserted foreign genes. The vector contains the lambda PL promoter PL, operator OL and two utilization sites (NutL and NutR) to relieve transcriptional polarity effects when N protein is provided (Gross et al., 1985. Mol. & Cell. Biol. 5:1015).

The plasmid expressing the pD-PRAME fusion protein was designed so the PRAME amino acids were added to the C-terminal of a 108 amino acids derivative of pD without its signal sequence (secretion or signal sequence) (i.e. residues 20-127). To this construction, three unrelated amino acids (Met and Asp and a Proline) were added at the N-terminal of the derivative of pD, and for certain constructions a his tail at the C-terminal of the PRAME amino acids was included (see table A above). This construct could alternatively be described as containing 109 amino acids derivative of pD, if the N-terminal Met is considered to come from the pD sequence.

### Host Strain and Transformation

Hosts from E. Coli strain AR58 (Mott et al, Proc. Natl. Acad. Sci. USA, vol 82, pp 88-92, January 1985, Biochemistry) were transformed with plasmid DNA for Examples/constructs 1 and 2.

The AR58 lysogenic E. coli strain used for the production of Examples/constructs 1 and 2 is a derivative of the standard NIH E.coli K12 strain N99 (F-su-gaIK2, lacZ-thr-). It contains a defective lysogenic lambda phage (galE::TN10, 1 Kil-cI857 DH1). The Kil-phenotype prevents the shut off of host macromolecular synthesis. The cI857 mutation confers a temperature sensitive lesion to the cI repressor. The DH1 deletion removes the lambda phage right operon and the hosts bio, uvr3, and ch1A loci. The AR58 strain was generated by transduction of N99 with a P lambda phage stock previously grown on an SA500 derivative (galE::TN10, 1 Kil-cI857 DH1). The introduction of the defective lysogen into N99 was selected with tetracycline by virtue of the presence of a TN10 transposon coding for tetracyclin resistance in the adjacent galE gene. N99 and SA500 are E.coli K12 strains derived from Dr. Martin Rosenberg's laboratory at the National Institutes of Health.

Vectors containing the PL promoter, are introduced into an E. coli lysogenic host to stabilize the plasmid DNA. Lysogenic host strains contain replication-defective lambda phage DNA integrated into the genome (Shatzman et al., 1983; In Experimental Manipulation of Gene Expression. Inouya (ed) pp 1-14. Academic Press NY). The lambda phage DNA directs the synthesis of the cI repressor protein which binds to the OL repressor of the vector and prevents binding of RNA polymerase to the PL promoter and thereby transcription of the inserted gene. The cI gene of the expression strain AR58 contains a temperature sensitive mutation so that PL directed transcription can be regulated by temperature shift, i.e. an increase in culture temperature inactivates the repressor and synthesis of the foreign protein is initiated. This expression system allows controlled synthesis of foreign proteins especially of those that may be toxic to the cell (Shimataka & Rosenberg, 1981. Nature 292:128).

Hosts from E. Coli strain BLR (DE3) Novagen, Wis., USA (catalogue number: 69053-4) BLR (DE3) Novagen, Wis., USA (catalogue number: 69053-4) BLR is a recA-derivative of BL21 that improves plasmid monomer yields and may help stabilize target plasmids containing repetitive sequences or whose products may cause the loss of the DE3 prophage (1, 2) were transformed with plasmid DNA from examples/constructs 3 and 4.

Each of transformation was carried out by standard methods with CaCl₂-treated cells (Hanahan D. <<Plasmid transformation by Simanis.>> In Glover, D. M. (Ed), DNA cloning. IRL Press London. (1985): p. 109-135.).

### Growth and Induction of Bacterial Host Strain

Culture

Bacteria were grown-on 20 ml of Luria-Bertani (LB) broth (BD)+1% (w/v) glucose (Laboratoire MAT, catalogue number: GR-0101)+antibiotic(Carbenicillin 100 μg/ml for pET21b, kanamycin 40 μg/ml for TCMP14). Cultures were incubated at 33° C., for AR58 cells and at 37° C., for BLR (DE3) cells until an O.D._{600 nm} around 0.8.

Induction

At O.D._{600 nm} around 0.8, the cultures BLR (DE3) were induced at 1 mM isopropyl β-D-1-thiogalactopyranoside (IPTG; EMD Chemicals Inc., catalogue number: 5815) and incubated for 2 hours or 3 hours at 37° C. although solubility may be increased if a lower temperature is used.

At O.D._{600 nm} around 0.8, the cultures AR58 were induced by heat activation at 37° C. and incubated for 7 hours.

The bacterial growth was adequate for the two expression systems.

Extraction and Purification of the Protein

Upon expression of the polypeptide in culture, cells are typically harvested by centrifugation then disrupted by physical or chemical means (if the expressed polypeptide is not secreted into the media) and the resulting crude extract retained to isolate the polypeptide of interest. BugBuster™ Protein Extraction Reagent is used under conditions recommended by the suppliers (Novagen).

### PD⅓-Prame-His Protein Purirication

E. coli cell paste was resuspended in 20 mM Tris buffer pH 8.5 then passed through homogenizer system (Panda from Niro Soavi S.p.A.-2 passes-750 bars). After addition of 2 mM MgCl₂ and Benzonase (50 U/ml), homogenate was incubated 1 hour at room temperature (RT) under gentle agitation then centrifuged 30 minutes at 15900 g and RT. Resulting pellet was resuspended in 20 mM Tris buffer pH 8.5 containing 1% Sodium Dodecyl Sulphate (SDS) and 60 mM Glutathione and incubated 30 minutes at RT under gentle agitation. After centrifugation 30 minutes at 15900 g and RT, pellet was discarded.

Centrifugation supernatant was 10-fold diluted in 20 mM Tris buffer containing 6.66 M Urea, 0.333 M sodium chloride (NaCl) and 11.11 mM Imidazole and then subjected to chromatographic separation on a Nickel ion metal affinity column (IMAC Sepharose 6 FF-GE Healthcare) equilibrated in a 20 mM Tris buffer pH 8.5 containing 0.1% SDS, 6.0 M Urea, 0.3 M NaCl and 10 mM Imidazole. After washing of the column with 20 mM Tris buffer pH 8.5 containing 0.5% Sarcosyl, 6.0 M Urea, 0.3 M NaCl and 10 mM Imidazole, antigen was eluted from the column by increasing the concentration of Imidazole up to 40 mM in the same washing buffer. After addition of phosphate up to 50 mM, antigen positive eluate was passed through a Macro-Prep Ceramic Hydroxyapatite type II column (Bio-Rad) equilibrated in a 20 mM Tris buffer pH 8.5 containing 50 mM phosphate, 0.5% Sarcosyl, 6.0 M Urea and 0.3 M NaCl. Hydroxyapatite flow-through containing the antigen was then diafiltered against 5 mM Borate buffer pH 9.8 containing 3.15% Sucrose on an Omega 30 kDa membrane (Pall). Ultrafiltration retentate was sterilized by filtration through a 0.45/0.22 μm Cellulose acetate membrane (Sartorius). Purified material was stored at −70° C.

An alternative purification process has also been used, which differs from the above process in the following steps:
No benzonase treatment
No shift from SDS to sarcosyl on IMAC column (SDS from extraction up to HA step)
The buffer used for the diafiltration was 5 mM Tris buffer pH 8.5-0.5 M Arginine. This alternative purification process resulted in incomplete SDS removal with residual value around 0.05 and 0.085%.

Purification

The expressed recombinant proteins were purified from supernatant fractions obtained after centrifugation of induced E. coli using a His-Bind metal chelation resin (QIAgen, Chatsworth, Calif.) according to the instructions from the resin manufacturer.

### Characterization of the Protein

### SDS-Page:

Gel: NuPAGE 4-12% Bis-Tris Gel 1.0 mm 15 or 26 wells (Invitrogen catalog number: NP0323BOX)

See FIGS. 1 and 2 below, which show SDS page analysis of Example 3 and 4 and 3a and 4a respectively, wherein the different recombinant ⅓pD-PRAME proteins with or without his-tag migrate on gel at an apparent molecular weight of ˜70 kDa. The recombinant proteins are found as an inclusion bodies in the E coli cell lysate, after induction.

Preparation of samples, buffers and migration conditions were done under conditions recommended by the suppliers (Invitrogen). 10 μl of all preparations were loaded (before induction (BI) and after induction (AI)) in wells corresponding to 100 μl of culture equivalent.

### Western Blot

Membranes were blocked for 30 minutes at 37° C., 60 RPM using 3% milk/PBS 1× fresh solution. After the blocking incubation, primary antibodies were added (rabbit anti-PRAME; GSK Biologicals SA) at dilution 1:5000 or (α-6× His tag (AbCam) at dilution 1:3000 in 3% milk/PBS 1× fresh solution for 1 hour at 37° C., 60 RPM. After that, membranes were washed three times 5 minutes at room temperature using 0.02% Tween20/PBS 1×. Secondary antibodies were added (perox donkey anti-IgG (H+L) rabbit (Jackson laboratory) at dilution 1:20 000 using 3% milk/PBS 1× fresh solution. Membranes were incubated for 1 hour at 37° C., 60 RPM. After that, membranes were washed three times 5 minutes at room temperature using 0.02% Tween20/PBS 1× before the membrane expositions to peroxydase substrate (KH₂PO₄, 10 mM; (NH₄)₂SO₄, 10 mM; O-dianisidine, 0.01% & hydrogen peroxide 0.045%) or alkaline phosphatase substrate (Sigma Fast) following the supplier's recommendations.

### Molecular Analysis:

### Example/Construct 1

| Analysis | Entire Protein |
|---|---|
| Length | 629 aa |
| Molecular Weight | 71629.96 m.w. |
| 1 microgram = | 13.961 pMoles |
| Molar Extinction | 67680 |
| coefficient | |
| 1 A[280] corr. to | 1.06 mg/ml |
| A[280] of 1 mg/ml | 0.94 AU |
| Isoelectric Point | 6.41 |
| Charge at pH 7 | −5.84 |

### Example/Construct 2

| Analysis | Entire Protein |
|---|---|
| Length | 620 aa |
| Molecular Weight | 70561.90 m.w. |
| 1 microgram = | 14.172 pMoles |
| Molar Extinction | 67680 |
| coefficient | |
| 1 A[280] corr. to | 1.04 mg/ml |
| A[280] of 1 mg/ml | 0.96 AU |
| Isoelectric Point | 6.28 |
| Charge at pH 7 | −6.36 |

### Example/Construct 3

| Analysis | Entire Protein |
|---|---|
| Length | 628 aa |
| Molecular Weight | 71627.01 m.w. |
| 1 microgram = | 13.961 pMoles |
| Molar Extinction | 67680 |
| coefficient | |
| 1 A[280] corr. to | 1.06 mg/ml |
| A[280] of 1 mg/ml | 0.94 AU |
| Isoelectric Point | 6.34 |
| Charge at pH 7 | −6.84 |

### Example/Construct 4

| Analysis | Entire Protein |
|---|---|
| Length | 620 aa |
| Molecular Weight | 70561.90 m.w. |
| 1 microgram = | 14.172 pMoles |
| Molar Extinction | 67680 |
| coefficient | |
| 1 A[280] corr. to | 1.04 mg/ml |
| A[280] of 1 mg/ml | 0.96 AU |
| Isoelectric Point | 6.28 |
| Charge at pH 7 | −6.36 |

### Example 5

Evaluation of the Production of Proteins With or Without the Secretion Signal (Secretion or Signal Sequence) of the Protein D ⅓ in the fusion protein.

TABLE B

| Protein | Expression level |
|---|---|
| PD1/3-PRAME with secretion signal | + |
| PD1/3-PRAME without secretion signal | +++ |

FIG. 11: SDS page analysis after Coomassie-blue staining of recombinant ⅓PD-PRAME with or without secretion signal after IPTG induction of E. coli BL21 DE3 strain transformed with recombinant pET21. An equivalent of 100 μL of culture after 3 hours of induction in BL21 DE3 strain with 1 mM IPTG at 37° C. has been loaded on gel. Those constructs are presented on gel before (BI) and after (AI) inductions in soluble (supernatant) and non-soluble (pellet) fractions. Lane 1: Standard Broad Range prestain (BioRad Cat#161-0318), lane 2 (pD⅓-PRAME+SS, BI, supernatant), lane 3 (pD⅓-PRAME+SS, BI, pellet), lane 4 (pD⅓-PRAME+SS, AI, supernatant), lane 5 (pD⅓-PRAME+SS, AI, pellet), lane 6 (pD⅓-PRAME+SS+His, BI, supernatant), lane 7 (pD⅓-PRAME+SS+His, BI, pellet), lane 8 (pD⅓-PRAME+SS+His, AI, supernatant), lane 9 (pD⅓-PRAME+SS+His, AI, pellet), lane 10 (pD⅓-PRAME w/o SS, BI, supernatant), lane 11 (pD⅓-PRAME w/o SS, BI, pellet), lane 12 (pD⅓-PRAME w/o SS, AI, supernatant), lane 13 (pD⅓-PRAME w/o SS, AI, pellet), lane 14 (pD⅓-PRAME w/o SS+His, BI, supernatant), lane 15 (pD⅓-PRAME w/o SS+His, BI, pellet), lane 16 (pD⅓-PRAME w/o SS+His, AI, supernatant), lane 17 (pD⅓-PRAME w/o SS+His, AI, pellet).

### Example 6

### Immunogenicity of PD-PRAME-His Formulated in AS01B or AS15: Dose Range of Antigen in a Constant Dose of Adjuvant.

Aim: dose-range of antigen to select the best dose to use in preclinical experiments

### Protocol:

6 groups of 12 CB6F1 mice received intra-muscular (IM) injections at day 0 and 14 of:
1. PBS
2. PRAME (50*μg) in AS01B or AS15
3. PRAME (10 μg) in AS01B or AS15
4. PRAME (2 μg) in AS01B or AS15
5. PRAME (0.4 μg) in AS01B or AS15
6. PRAME (0.08 μg) in AS01B or AS15

44.7 μg actually administered instead of the 50 μg intended dose

AS01B is a liposomal adjuvant formulation comprising QS21 and 3D-MPL; AS15 is a liposomal adjuvant formulation comprising QS21, 3D-MPL and CpG.

The construct used in this example was Example/Construct 3a (pET26 with a His tail), provided in 5 mM Tris buffer pH 8.5-0.5 M Arginine. Protein provided in a borate buffer with sucrose may also be used.

### Read-Outs:

Intracellular Cytokine Staining (ICS) 14 days post 2 injections after in vitro restimulation of spleen cells (4 pools of 3 mice per group) with the pool of peptides PRAME at 1 μg/ml/peptide (15-mer)

### CD4 Response (AS01B Adjuvant)

Results of ICS for CD4 cytokines for the AS01B adjuvant are shown in FIG. 3. In this experiment it may be concluded that the best dose of PRAME antigen to induce a CD4 response in AS01B under these conditions seems to be 2 μg.

### CD8 Response (AS01B Adjuvant)

Results of ICS for CD8 cytokines for the AS01B adjuvant are shown in FIG. 4. The data appear to show a very low CD8 response and heterogeneity of the response intra-group. CD4 response (AS15 adjuvant)

Results of ICS for CD4 cytokines for the AS15 adjuvant are shown in FIG. 5. These data appear to show that a similar CD4 response was induced with 44 μg, 10 μg, 2 μg and 0.4 μg of PRAME formulated in AS15; with a decreased response induced with 0.08 μg PRAME

### CD8 Response (AS15 Adjuvant)

Results of ICS for CD8 cytokines for the AS15 adjuvant are shown in FIG. 6. These data appear to show no CD8 response (background in the PBS group)

### Example 7

In summary, for the inventions described herein, the following summary may be used to described specific constructs of PD⅓-PRAME that have so far been generated:

### Constructs Used for PD⅓-PRAME

No signal sequence of Protein D are included (amino acids 2 to 19 of protein D)

The Methionine of Protein D is included (AA 1 of the protein D)

Two unrelated AA (Asp and Pro) are substituted for amino acids 2-Lys and 3-Leu of Protein D

The first 109 AA of protein D after the signal sequence of protein D are included (109 amino acids including the first Met in N-term+AA20 to 127 of the protein D)

AA 1-509 of PRAME are included (full length original sequence of PRAME)

With or without a His tail composed of one of the following:
Three unrelated amino acid (Thr, Ser and Gly)+6 His residues for cloning in TCMP14 plasmid; or
Two unrelated amino acid (Leu and Glu)+6 His residues for cloning in pET21 plasmid; or
6 His residues for cloning in pET26 plasmid.
   pD⅓-PRAME±His Tail Protein:

| | pD ⅓ | PRAME | |
|---|---|---|---|
| | 20-127 | 1-509 | |
| N term MDP | pD ⅓ | PRAME | C term |
| N term MDP | pD ⅓ | PRAME | TSG 6xHis (in TCMP14) C term |
| N term MDP | pD ⅓ | PRAME | LE 6xHis (in pET21) C term |
| N term MDP | pD ⅓ | PRAME | 6xHis (in pET26) C term |

A marked up amino acid sequence of examples of constructs of the present invention is shown in FIG. 7

Alignments of the following constructs are shown in FIGS. 8, 9 and 10:

Alignment between LipoD-MAGE3-His and D⅓-PRAME-His (FIG. 8)

Alignment between the shared sequence of the original protein D from Haemophilus influenzae and the LipoD-MAGE3-His (FIG. 9)

Alignment between the shared sequence of the original protein D from Haemophilus influenzae, the LipoD-MAGE3-His and the pD⅓-PRAME-His (FIG. 10)

Formulation of Vaccine Preparation Using Fusion Proteins:

The fusion proteins of the invention can be formulated into vaccines which are either adjuvanted or not. In one embodiment, as an adjuvant, the formulation may comprise a mixture of 3de-O-acylated monophosphoryl lipid A (3D-MPL) and QS21 in an oil/water emulsion. The adjuvant system SBAS2 has been previously described WO 95/17210. The adjuvant for use in the present invention may alternatively comprise 3de-O-acylated monophosphoryl lipid A (3D-MPL), QS21 and CpG in an oil-in water formulation or in a liposomal formulation.

3D-MPL: is an immunostimulant derived from the lipopolysaccharide (LPS) of the Gram-negative bacterium Salmonella minnesota. MPL has been deacylated and is lacking a phosphate group on the lipid A moiety. This chemical treatment dramatically reduces toxicity while preserving the immunostimulant properties (Ribi, 1986).

It is believed that 3D-MPL combined with various vehicles may strongly enhance both the humoral and a TH1 type of cellular immunity.

QS21: is a natural saponin molecule extracted from the bark of the South American tree Quillaja saponaria Molina. A purification technique developed to separate the individual saponines from the crude extracts of the bark, permitted the isolation of the particular saponin, QS21, which is a triterpene glycoside demonstrating stronger adjuvant activity and lower toxicity as compared with the parent component. QS21 has been shown to activate MHC class I restricted CTLs to several subunit Ags, as well as to stimulate Ag specific lymphocytic proliferation (Kensil, 1992).

It is thought that there may be a synergistic effect of combinations of MPL and QS21 in the induction of both humoral and TH1 type cellular immune responses.

The oil/water emulsion comprises an organic phase made of 2 oils (a tocopherol and squalene), and an aqueous phase of PBS containing Tween 80 as emulsifier. The emulsion comprised 5% squalene 5% tocopherol 0.4% Tween 80 and had an average particle size of 180 nm and is known as SB62 (see WO 95/17210). The resulting oil droplets should have a size of approximately 180 nm.

The adjuvant for use in the present invention may be formulated as a combination of MPL and QS21, in an oil/water emulsion or in a liposomal formulation. This preparation should be delivered in vials of 0.7 ml to be admixed with lyophilized antigen or fusion protein (vials containing from 30 to 300 μg antigen).

Immunostimulatory oligonucleotides may also be used. Examples oligonucleotides for use in adjuvants or vaccines of the present invention include CpG containing oligonucleotides, generally containing two or more dinucleotide CpG motifs separated by at least three, more often at least six or more nucleotides. A CpG motif is a cytosine nucleotide followed by a guanine nucleotide. The CpG oligonucleotides are typically deoxynucleotides. In one embodiment the intemucleotide in the oligonucleotide is phosphorodithioate, or more preferably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. No. 5,666,153, U.S. Pat. No. 5,278,302 and WO 95/26204.

Examples of oligonucleotides are as follows:

TCC ATG ACG TTC CTG ACG TT (CpG 1826; SEQ ID
NO: 36)
TCT CCC AGC GTG CGC CAT (CPG 1758; SEQ ID NO: 37)
ACC GAT GAC GTC GCC GGT GAC GGC ACC ACG TCG TCG
TTT TGT CGT TTT GTC GTT (CpG 2006; SEQ ID NO: 38)
TCC ATG ACG TTC CTG ATG CT (CpG 1668; SEQ ID
NO: 39)
TCG ACG TTT TCG GCG CGC GCC G (CpG 5456; SEQ ID
NO: 40),

the sequences may contain phosphorothioate modified intemucleotide linkages.

Alternative CpG oligonucleotides may comprise one or more sequences above in that they have inconsequential deletions or additions thereto.

The CpG oligonucleotides may be synthesized by any method known in the art (for example see EP 468520). Conveniently, such oligonucleotides may be synthesized utilizing an automated synthesizer.

In one embodiment of the present invention an adjuvant combination for use in the invention includes one or more of the following components: 3D-MPL and QS21 (EP 0 671 948 B1); oil in water emulsions comprising 3D-MPL and QS21 (WO 95/17210, WO 98/56414); or 3D-MPL formulated with other carriers (EP 0 689 454 B1). Other adjuvant systems that may be used in the present invention comprise a combination of 3D-MPL, QS21 and a CpG oligonucleotide as described in U.S. Pat. No. 6,558,670 and U.S. Pat. No. 6,544,518.

The final vaccine may be obtained after reconstitution of the lyophilized formulation.

### REFERENCES

1. A. Roca (U. of Wisconsin), personal communication.
2. Studier, F. W. (1991) J. Mol. Biol. 219, 37-44.
3. Jan H. Kessler^{a} et al The Journal of Experimental Medicine, Volume 193, Number 1, Jan. 1, 2001 73-88,
4. Ikeda H et al Immunity, February; 6(2): 1997, 199-208

SEQ ID NO: 1

DNA sequence for Example 1
atggatccaagcagccattcatcaaatatggcgaatacccaaatgaaatc
agacaaaatcattattgctcaccgtggtgctagcggttatttaccagagc
atacgttagaatctaaagcacttgcgtttgcacaacaggctgattattta
gagcaagatttagcaatgactaaggatggtcgtttagtggttattcacga
tcactttttagatggcttgactgatgttgcgaaaaaattcccacatcgtc
atcgtaaagatggccgttactatgtcatcgactttaccttaaaagaaatt
caaagtttagaaatgacagaaaactttgaaaccatggaacgaaggcgttt
gtggggttccattcagagccgatacatcagcatgagtgtgtggacaagcc
cacggagacttgtggagctggcagggcagagcctgctgaaggatgaggcc
ctggccattgccgccctggagttgctgcccagggagctcttcccgccact
cttcatggcagcctttgacgggagacacagccagaccctgaaggcaatgg
tgcaggcctggcccttcacctgcctccctctgggagtgctgatgaaggga
caacatcttcacctggagaccttcaaagctgtgcttgatggacttgatgt
gctccttgcccaggaggttcgccccaggaggtggaaacttcaagtgctgg
atttacggaagaactctcatcaggacttctggactgtatggtctggaaac
agggccagtctgtactcatttccagagccagaagcagctcagcccatgac
aaagaagcgaaaagtagatggtttgagcacagaggcagagcagcccttca
ttccagtagaggtgctcgtagacctgttcctcaaggaaggtgcctgtgat
gaattgttctcctacctcattgagaaagtgaagcgaaagaaaaatgtact
acgcctgtgctgtaagaagctgaagatttttgcaatgcccatgcaggata
tcaagatgatcctgaaaatggtgcagctggactctattgaagatttggaa
gtgacttgtacctggaagctacccaccttggcgaaattttctccttacct
gggccagatgattaatctgcgtagactcctcctctcccacatccatgcat
cttcctacatttccccggagaaggaagagcagtatatcgcccagttcacc
tctcagttcctcagtctgcagtgcctgcaggctctctatgtggactcttt
atttttccttagaggccgcctggatcagttgctcaggcacgtgatgaacc
ccttggaaaccctctcaataactaactgccggctttcggaaggggatgtg
atgcatctgtcccagagtcccagcgtcagtcagctaagtgtcctgagtct
aagtggggtcatgctgaccgatgtaagtcccgagcccctccaagctctgc
tggagagagcctctgccaccctccaggacctggtctttgatgagtgtggg
atcacggatgatcagctccttgccctcctgccttccctgagccactgctc
ccagcttacaaccttaagcttctacgggaattccatctccatatctgcct
tgcagagtctcctgcagcacctcatcgggctgagcaatctgacccacgtg
ctgtatcctgtccccctggagagttatgaggacatccatggtaccctcca
cctggagaggcttgcctatctgcatgccaggctcagggagttgctgtgtg
agttggggcggcccagcatggtctggcttagtgccaacccctgtcctcac
tgtggggacagaaccttctatgacccggagcccatcctgtgcccctgttt
catgcctaacactagtggccaccatcaccatcaccat

SEQ ID NO: 2

Amino Acid Sequence for Example 1
MDPSSHSSNMANTQMKSDKIIIAHRGASGYLPEHTLESKALAFAQQADYL
EQDLAMTKDGRLVVIHDHFLDGLTDVAKKFPHRHRKDGRYYVIDFTLKEI
QSLEMTENFETMERRRLWGSIQSRYISMSVWTSPRRLVELAGQSLLKDEA
LAIAALELLPRELFPPLFMAAFDGRHSQTLKAMVQAWPFTCLPLGVLMKG
QHLHLETFKAVLDGLDVLLAQEVRPRRWKLQVLDLRKNSHQDFWTVWSGN
RASLYSFPEPEAAQPMTKKRKVDGLSTEAEQPFIPVEVLVDLFLKEGACD
ELFSYLIEKVKRKKNVLRLCCKKLKIFAMPMQDIKMILKMVQLDSIEDLE
VTCTWKLPTLAKFSPYLGQMINLRRLLLSHIHASSYISPEKEEQYIAQFT
SQFLSLQCLQALYVDSLFFLRGRLDQLLRHVMNPLETLSITNCRLSEGDV
MHLSQSPSVSQLSVLSLSGVMLTDVSPEPLQALLERASATLQDLVFDECG
ITDDQLLALLPSLSHCSQLTTLSFYGNSISISALQSLLQHLIGLSNLTHV
LYPVPLESYEDIHGTLHLERLAYLHARLRELLCELGRPSMVWLSANPCPH
CGDRTFYDPEPILCPCFMPNTSGHHHHHH

SEQ ID NO: 3

DNA Sequence for example 2
atggatccaagcagccattcatcaaatatggcgaatacccaaatgaaatc
agacaaaatcattattgctcaccgtggtgctagcggttatttaccagagc
atacgttagaatctaaagcacttgcgtttgcacaacaggctgattattta
gagcaagatttagcaatgactaaggatggtcgtttagtggttattcacga
tcactttttagatggcttgactgatgttgcgaaaaaattcccacatcgtc
atcgtaaagatggccgttactatgtcatcgactttaccttaaaagaaatt
caaagtttagaaatgacagaaaactttgaaaccatggaacgaaggcgttt
gtggggttccattcagagccgatacatcagcatgagtgtgtggacaagcc
cacggagacttgtggagctggcagggcagagcctgctgaaggatgaggcc
ctggccattgccgccctggagttgctgcccagggagctcttcccgccact
cttcatggcagcctttgacgggagacacagccagaccctgaaggcaatgg
tgcaggcctggcccttcacctgcctccctctgggagtgctgatgaaggga
caacatcttcacctggagaccttcaaagctgtgcttgatggacttgatgt
gctccttgcccaggaggttcgccccaggaggtggaaacttcaagtgctgg
atttacggaagaactctcatcaggacttctggactgtatggtctggaaac
agggccagtctgtactcatttccagagccagaagcagctcagcccatgac
aaagaagcgaaaagtagatggtttgagcacagaggcagagcagcccttca
ttccagtagaggtgctcgtagacctgttcctcaaggaaggtgcctgtgat
gaattgttctcctacctcattgagaaagtgaagcgaaagaaaaatgtact
acgcctgtgctgtaagaagctgaagatttttgcaatgcccatgcaggata
tcaagatgatcctgaaaatggtgcagctggactctattgaagatttggaa
gtgacttgtacctggaagctacccaccttggcgaaattttctccttacct
gggccagatgattaatctgcgtagactcctcctctcccacatccatgcat
cttcctacatttccccggagaaggaagagcagtatatcgcccagttcacc
tctcagttcctcagtctgcagtgcctgcaggctctctatgtggactcttt
atttttccttagaggccgcctggatcagttgctcaggcacgtgatgaacc
ccttggaaaccctctcaataactaactgccggctttcggaaggggatgtg
atgcatctgtcccagagtcccagcgtcagtcagctaagtgtcctgagtct
aagtggggtcatgctgaccgatgtaagtcccgagcccctccaagctctgc
tggagagagcctctgccaccctccaggacctggtctttgatgagtgtggg
atcacggatgatcagctccttgccctcctgccttccctgagccactgctc
ccagcttacaaccttaagcttctacgggaattccatctccatatctgcct
tgcagagtctcctgcagcacctcatcgggctgagcaatctgacccacgtg
ctgtatcctgtccccctggagagttatgaggacatccatggtaccctcca
cctggagaggcttgcctatctgcatgccaggctcagggagttgctgtgtg
agttggggcggcccagcatggtctggcttagtgccaacccctgtcctcac
tgtggggacagaaccttctatgacccggagcccatcctgtgcccctgttt
catgcctaac

SEQ ID NO: 4

AMINO ACIDS SEQUENCE EXAMPLE 2
MDPSSHSSNMANTQMKSDKIIIAHRGASGYLPEHTLESKALAFAQQADYL
EQDLAMTKDGRLVVIHDHFLDGLTDVAKKFPHRHRKDGRYYVIDFTLKEI
QSLEMTENFETMERRRLWGSIQSRYISMSVWTSPRRLVELAGQSLLKDEA
LAIAALELLPRELFPPLFMAAFDGRHSQTLKAMVQAWPFTCLPLGVLMKG
QHLHLETFKAVLDGLDVLLAQEVRPRRWKLQVLDLRKNSHQDFWTVWSGN
RASLYSFPEPEAAQPMTKKRKVDGLSTEAEQPFIPVEVLVDLFLKEGACD
ELFSYLIEKVKRKKNVLRLCCKKLKIFAMPMQDIKMILKMVQLDSIEDLE
VTCTWKLPTLAKFSPYLGQMINLRRLLLSHIHASSYISPEKEEQYIAQFT
SQFLSLQCLQALYVDSLFFLRGRLDQLLRHVMNPLETLSITNCRLSEGDV
MHLSQSPSVSQLSVLSLSGVMLTDVSPEPLQALLERASATLQDLVFDECG
ITDDQLLALLPSLSHCSQLTTLSFYGNSISISALQSLLQHLIGLSNLTHV
LYPVPLESYEDIHGTLHLERLAYLHARLRELLCELGRPSMVWLSANPCPH
CGDRTFYDPEPILCPCFMPN

SEQ ID NO:5

DNA sequence for Example 3
atggatccaagcagccattcatcaaatatggcgaatacccaaatgaaatc
agacaaaatcattattgctcaccgtggtgctagcggttatttaccagagc
atacgttagaatctaaagcacttgcgtttgcacaacaggctgattattta
gagcaagatttagcaatgactaaggatggtcgtttagtggttattcacga
tcactttttagatggcttgactgatgttgcgaaaaaattcccacatcgtc
atcgtaaagatggccgttactatgtcatcgactttaccttaaaagaaatt
caaagtttagaaatgacagaaaactttgaaaccatggaacgaaggcgttt
gtggggttccattcagagccgatacatcagcatgagtgtgtggacaagcc
cacggagacttgtggagctggcagggcagagcctgctgaaggatgaggcc
ctggccattgccgccctggagttgctgcccagggagctcttcccgccact
cttcatggcagcctttgacgggagacacagccagaccctgaaggcaatgg
tgcaggcctggcccttcacctgcctccctctgggagtgctgatgaaggga
caacatcttcacctggagaccttcaaagctgtgcttgatggacttgatgt
gctccttgcccaggaggttcgccccaggaggtggaaacttcaagtgctgg
atttacggaagaactctcatcaggacttctggactgtatggtctggaaac
agggccagtctgtactcatttccagagccagaagcagctcagcccatgac
aaagaagcgaaaagtagatggtttgagcacagaggcagagcagcccttca
ttccagtagaggtgctcgtagacctgttcctcaaggaaggtgcctgtgat
gaattgttctcctacctcattgagaaagtgaagcgaaagaaaaatgtact
acgcctgtgctgtaagaagctgaagatttttgcaatgcccatgcaggata
tcaagatgatcctgaaaatggtgcagctggactctattgaagatttggaa
gtgacttgtacctggaagctacccaccttggcgaaattttctccttacct
gggccagatgattaatctgcgtagactcctcctctcccacatccatgcat
cttcctacatttccccggagaaggaagagcagtatatcgcccagttcacc
tctcagttcctcagtctgcagtgcctgcaggctctctatgtggactcttt
atttttccttagaggccgcctggatcagttgctcaggcacgtgatgaacc
ccttggaaaccctctcaataactaactgccggctttcggaaggggatgtg
atgcatctgtcccagagtcccagcgtcagtcagctaagtgtcctgagtct
aagtggggtcatgctgaccgatgtaagtcccgagcccctccaagctctgc
tggagagagcctctgccaccctccaggacctggtctttgatgagtgtggg
atcacggatgatcagctccttgccctcctgccttccctgagccactgctc
ccagcttacaaccttaagcttctacgggaattccatctccatatctgcct
tgcagagtctcctgcagcacctcatcgggctgagcaatctgacccacgtg
ctgtatcctgtccccctggagagttatgaggacatccatggtaccctcca
cctggagaggcttgcctatctgcatgccaggctcagggagttgctgtgtg
agttggggcggcccagcatggtctggcttagtgccaacccctgtcctcac
tgtggggacagaaccttctatgacccggagcccatcctgtgcccctgttt
catgcctaacctcgagcaccaccaccaccaccac

SEQ ID NO: 6

Amino acids sequence: for Example 3
MDPSSHSSNMANTQMKSDKIIIAHRGASGYLPEHTLESKALAFAQQADYL
EQDLAMTKDGRLVVIHDHFLDGLTDVAKKFPHRHRKDGRYYVIDFTLKEI
QSLEMTENFETMERRRLWGSIQSRYISMSVWTSPRRLVELAGQSLLKDEA
LAIAALELLPRELFPPLFMAAFDGRHSQTLKAMVQAWPFTCLPLGVLMKG
QHLHLETFKAVLDGLDVLLAQEVRPRRWKLQVLDLRKNSHQDFWTVWSGN
RASLYSFPEPEAAQPMTKKRKVDGLSTEAEQPFIPVEVLVDLFLKEGACD
ELFSYLIEKVKRKKNVLRLCCKKLKIFAMPMQDIKMILKMVQLDSIEDLE
VTCTWKLPTLAKFSPYLGQMINLRRLLLSHIHASSYISPEKEEQYIAQFT
SQFLSLQCLQALYVDSLFFLRGRLDQLLRHVMNPLETLSITNCRLSEGDV
MHLSQSPSVSQLSVLSLSGVMLTDVSPEPLQALLERASATLQDLVFDECG
ITDDQLLALLPSLSHCSQLTTLSFYGNSISISALQSLLQHLIGLSNLTHV
LYPVPLESYEDIHGTLHLERLAYLHARLRELLCELGRPSMVWLSANPCPH
CGDRTFYDPEPILCPCFMPNLEHHHHHH

SEQ ID NO: 7.

DNA sequence for Example 4:
atggatccaagcagccattcatcaaatatggcgaatacccaaatgaaatc
agacaaaatcattattgctcaccgtggtgctagcggttatttaccagagc
atacgttagaatctaaagcacttgcgtttgcacaacaggctgattattta
gagcaagatttagcaatgactaaggatggtcgtttagtggttattcacga
tcactttttagatggcttgactgatgttgcgaaaaaattcccacatcgtc
atcgtaaagatggccgttactatgtcatcgactttaccttaaaagaaatt
caaagtttagaaatgacagaaaactttgaaaccatggaacgaaggcgttt
gtggggttccattcagagccgatacatcagcatgagtgtgtggacaagcc
cacggagacttgtggagctggcagggcagagcctgctgaaggatgaggcc
ctggccattgccgccctggagttgctgcccagggagctcttcccgccact
cttcatggcagcctttgacgggagacacagccagaccctgaaggcaatgg
tgcaggcctggcccttcacctgcctccctctgggagtgctgatgaaggga
caacatcttcacctggagaccttcaaagctgtgcttgatggacttgatgt
gctccttgcccaggaggttcgccccaggaggtggaaacttcaagtgctgg
atttacggaagaactctcatcaggacttctggactgtatggtctggaaac
agggccagtctgtactcatttccagagccagaagcagctcagcccatgac
aaagaagcgaaaagtagatggtttgagcacagaggcagagcagcccttca
ttccagtagaggtgctcgtagacctgttcctcaaggaaggtgcctgtgat
gaattgttctcctacctcattgagaaagtgaagcgaaagaaaaatgtact
acgcctgtgctgtaagaagctgaagatttttgcaatgcccatgcaggata
tcaagatgatcctgaaaatggtgcagctggactctattgaagatttggaa
gtgacttgtacctggaagctacccaccttggcgaaattttctccttacct
gggccagatgattaatctgcgtagactcctcctctcccacatccatgcat
cttcctacatttccccggagaaggaagagcagtatatcgcccagttcacc
tctcagttcctcagtctgcagtgcctgcaggctctctatgtggactcttt
atttttccttagaggccgcctggatcagttgctcaggcacgtgatgaacc
ccttggaaaccctctcaataactaactgccggctttcggaaggggatgtg
atgcatctgtcccagagtcccagcgtcagtcagctaagtgtcctgagtct
aagtggggtcatgctgaccgatgtaagtcccgagcccctccaagctctgc
tggagagagcctctgccaccctccaggacctggtctttgatgagtgtggg
atcacggatgatcagctccttgccctcctgccttccctgagccactgctc
ccagcttacaaccttaagcttctacgggaattccatctccatatctgcct
tgcagagtctcctgcagcacctcatcgggctgagcaatctgacccacgtg
ctgtatcctgtccccctggagagttatgaggacatccatggtaccctcca
cctggagaggcttgcctatctgcatgccaggctcagggagttgctgtgtg
agttggggcggcccagcatggtctggcttagtgccaacccctgtcctcac
tgtggggacagaaccttctatgacccggagcccatcctgtgcccctgttt
catgcctaac

SEQ ID NO: 8

Amino Acid Sequence for Example 4
MDPSSHSSNMANTQMKSDKIIIAHRGASGYLPEHTLESKALAFAQQADYL
EQDLAMTKDGRLVVIHDHFLDGLTDVAKKFPHRHRKDGRYYVIDFTLKEI
QSLEMTENFETMERRRLWGSIQSRYISMSVWTSPRRLVELAGQSLLKDEA
LAIAALELLPRELFPPLFMAAFDGRHSQTLKAMVQAWPFTCLPLGVLMKG
QHLHLETFKAVLDGLDVLLAQEVRPRRWKLQVLDLRKNSHQDFWTVWSGN
RASLYSFPEPEAAQPMTKKRKVDGLSTEAEQPFIPVEVLVDLFLKEGACD
ELFSYLIEKVKRKKNVLRLCCKKLKIFAMPMQDIKMILKMVQLDSIEDLE
VTCTWKLPTLAKFSPYLGQMINLRRLLLSHIHASSYISPEKEEQYIAQFT
SQFLSLQCLQALYVDSLFFLRGRLDQLLRHVMNPLETLSITNCRLSEGDV
MHLSQSPSVSQLSVLSLSGVMLTDVSPEPLQALLERASATLQDLVFDECG
ITDDQLLALLPSLSHCSQLTTLSFYGNSISISALQSLLQHLIGLSNLTHV
LYPVPLESYEDIHGTLHLERLAYLHARLRELLCELGRPSMVWLSANPCPH
CGDRTFYDPEPILCPCFMPN

SEQ ID NO: 9

Codon optimised DNA sequence for example 3a
atggatccaagcagccattcatcaaatatggcgaatacccaaatgaaatc
agacaaaatcattattgctcaccgtggtgctagcggttatttaccagagc
atacgttagaatctaaagcacttgcgtttgcacaacaggctgattattta
gagcaagatttagcaatgactaaggatggtcgtttagtggttattcacga
tcactttttagatggcttgactgatgttgcgaaaaaattcccacatcgtc
atcgtaaagatggccgttactatgtcatcgactttaccttaaaagaaatt
caaagtttagaaatgacagaaaactttgaaaccatggaacgtcgtcgtct
gtggggcagcattcagagccgttatattagcatgagcgtgtggaccagcc
cgcgtcgtctggttgagctggccggccagagcctgctgaaagatgaagcg
ctggccattgcggcgctggagctgctgccgcgtgagctgtttccgccgct
gtttatggcggcgtttgatggccgtcatagccagaccctgaaagcgatgg
tgcaggcgtggccgtttacctgtctgccgctgggcgtgctgatgaaaggc
cagcatctgcatctggaaacctttaaagcggtgctggatggcctggatgt
gctgctggcccaggaagttcgtccgcgtcgttggaaactgcaagtgctgg
atctgcgtaaaaacagccatcaggatttttggaccgtgtggagcggcaat
cgtgcgagcctgtatagctttccggaaccggaagcggcgcagccgatgac
caaaaaacgtaaagtggatggcctgagcaccgaagcggaacagccgttta
ttccggtggaagtgctggttgacctgtttctgaaagaaggcgcctgcgac
gagctgtttagctatctgatcgaaaaagtgaaacgcaaaaaaaacgtgct
gcgtctgtgctgcaaaaaactgaaaatcttcgcgatgccgatgcaggata
ttaaaatgatcctgaaaatggtgcagctggatagcattgaggacctggaa
gtgacctgcacctggaaactgccgaccctggccaaatttagcccgtatct
gggccagatgattaacctgcgtcgtctgctgctgtctcatattcatgcga
gcagctatattagcccggaaaaagaagaacagtatatcgcgcagtttacc
agccagtttctgagcctgcaatgcctgcaagcgctgtatgtggatagcct
gttttttctgcgtggccgtctggatcagctgctgcgtcatgtgatgaatc
cgctggaaaccctgagcattaccaactgccgtctgagcgaaggcgatgtg
atgcatctgagccagagcccgagcgttagccagctgtctgttctgagcct
gagcggcgtgatgctgaccgatgtgagcccggaaccgctgcaagccctgc
tggaacgtgcgagcgcgaccctgcaagacctggtgtttgatgaatgcggc
attaccgatgatcagctgctggccctgctgccgagcctgagccattgcag
ccagctgaccaccctgagcttttatggcaacagcattagcattagcgcgc
tgcaaagcctgctgcaacatctgattggcctgagcaacctgacccatgtg
ctgtatccggtgccgctggaaagctatgaagatattcatggcaccctgca
tctggaacgtctggcctatctgcacgcgcgtctgcgtgagctgctgtgcg
agctgggccgtccgagcatggtttggctgtctgcgaatccgtgcccgcat
tgcggcgatcgtaccttttatgatccggaaccgattctgtgcccgtgctt
tatgccgaaccaccaccaccaccaccac

SEQ ID NO: 10

Amino Acid Sequence for Example 3a
MDPSSHSSNMANTQMKSDKIIIAHRGASGYLPEHTLESKALAFAQQADYL
EQDLAMTKDGRLVVIHDHFLDGLTDVAKKFPHRHRKDGRYYVIDFTLKEI
QSLEMTENFETMERRRLWGSIQSRYISMSVWTSPRRLVELAGQSLLKDEA
LAIAALELLPRELFPPLFMAAFDGRHSQTLKAMVQAWPFTCLPLGVLMKG
QHLHLETFKAVLDGLDVLLAQEVRPRRWKLQVLDLRKNSHQDFWTVWSGN
RASLYSFPEPEAAQPMTKKRKVDGLSTEAEQPFIPVEVLVDLFLKEGACD
ELFSYLIEKVKRKKNVLRLCCKKLKIFAMPMQDIKMILKMVQLDSIEDLE
VTCTWKLPTLAKFSPYLGQMINLRRLLLSHIHASSYISPEKEEQYIAQFT
SQFLSLQCLQALYVDSLFFLRGRLDQLLRHVMNPLETLSITNCRLSEGDV
MHLSQSPSVSQLSVLSLSGVMLTDVSPEPLQALLERASATLQDLVFDECG
ITDDQLLALLPSLSHCSQLTTLSFYGNSISISALQSLLQHLIGLSNLTHV
LYPVPLESYEDIHGTLHLERLAYLHARLRELLCELGRPSMVWLSANPCPH
CGDRTFYDPEPILCPCFMPNHHHHHH

SEQ ID NO: 11

Codon optimised DNA sequence for Example 4a
atggatccaagcagccattcatcaaatatggcgaatacccaaatgaaatc
agacaaaatcattattgctcaccgtggtgctagcggttatttaccagagc
atacgttagaatctaaagcacttgcgtttgcacaacaggctgattattta
gagcaagatttagcaatgactaaggatggtcgtttagtggttattcacga
tcactttttagatggcttgactgatgttgcgaaaaaattcccacatcgtc
atcgtaaagatggccgttactatgtcatcgactttaccttaaaagaaatt
caaagtttagaaatgacagaaaactttgaaaccatggaacgtcgtcgtct
gtggggcagcattcagagccgttatattagcatgagcgtgtggaccagcc
cgcgtcgtctggttgagctggccggccagagcctgctgaaagatgaagcg
ctggccattgcggcgctggagctgctgccgcgtgagctgtttccgccgct
gtttatggcggcgtttgatggccgtcatagccagaccctgaaagcgatgg
tgcaggcgtggccgtttacctgtctgccgctgggcgtgctgatgaaaggc
cagcatctgcatctggaaacctttaaagcggtcctggatggcctggatgt
gctgctggcccaggaagttcgtccgcgtcgttggaaactgcaagtgctgg
atctgcgtaaaaacagccatcaggatttttggaccgtgtggagcggcaat
cgtgcgagcctgtatagctttccggaaccggaagcggcgcagccgatgac
caaaaaacgtaaagtggatggcctgagcaccgaagcggaacagccgttta
ttccggtggaagtgctggttgacctgtttctgaaagaaggcgcctgcgac
gagctgtttagctatctgatcgaaaaagtgaaacgcaaaaaaaacgtgct
gcgtctgtgctgcaaaaaactgaaaatcttcgcgatgccgatgcaggata
ttaaaatgatcctgaaaatggtgcagctggatagcattgaggacctggaa
gtgacctgcacctggaaactgccgaccctggccaaatttagcccgtatct
gggccagatgattaacctgcgtcgtctgctgctgtctcatattcatgcga
gcagctatattagcccggaaaaagaagaacagtatatcgcgcagtttacc
agccagtttctgagcctgcaatgcctgcaagcgctgtatgtggatagcct
gttttttctgcgtggccgtctggatcagctgctgcgtcatgtgatgaatc
cgctggaaaccctgagcattaccaactgccgtctgagcgaaggcgatgtg
atgcatctgagccagagcccgagcgttagccagctgtctgttctgagcct
gagcggcgtgatgctgaccgatgtgagcccggaaccgctgcaagccctgc
tggaacgtgcgagcgcgaccctgcaagacctggtgtttgatgaatgcggc
attaccgatgatcagctgctggccctgctgccgagcctgagccattgcag
ccagctgaccaccctgagcttttatggcaacagcattagcattagcgcgc
tgcaaagcctgctgcaacatctgattggcctgagcaacctgacccatgtg
ctgtatccggtgccgctggaaagctatgaagatattcatggcaccctgca
tctggaacgtctggcctatctgcacgcgcgtctgcgtgagctgctgtgcg
agctgggccgtccgagcatggtttggctgtctgcgaatccgtgcccgcat
tgcggcgatcgtaccttttatgatccggaaccgattctgtgcccgtgctt
tatgccgaac

SEQ ID NO: 12

Amino Acid Sequence for Example 4a
MDPSSHSSNMANTQMKSDKIIIAHRGASGYLPEHTLESKALAFAQQADYL
EQDLAMTKDGRLVVIHDHFLDGLTDVAKKFPHRHRKDGRYYVIDFTLKEI
QSLEMTENFETMERRRLWGSIQSRYISMSVWTSPRRLVELAGQSLLKDEA
LAIAALELLPRELFPPLFMAAFDGRHSQTLKAMVQAWPFTCLPLQVLMKG
QHLHLETFKAVLDGLDVLLAQEVRPRRWKLQVLDLRKNSHQDFWTVWSGN
RASLYSFPEPEAAQPMTKKRKVDGLSTEAEQPFIPVEVLVDLFLKEGACD
ELFSYLIEKVKRKKNVLRLCCKKLKIFAMPMQDIKMILKMVQLDSIEDLE
VTCTWKLPTLAKFSPYLGQMINLRRLLLSHIHASSYISPEKEEQYTAQFT
SQFLSLQCLQALYVDSLFFLRGRLDQLLRHVMNPLETLSITNCRLSEGDV
MHLSQSPSVSQLSVLSLSGVMLTDVSPEPLQALLERASATLQDLVFDECG
ITDDQLLALLPSLSHCSQLTTLSFYGNSISISALQSLLQHLIGLSNLTHV
LYPVPLESYEDIHGTLHLERLAYLHALRLRELLCELGRPSMVWLSANPCP
HCGDRTFYDPEPILCPCFMPN

1. A fusion protein comprising:
(a) PRAME or an immunogenic fragment thereof, and
(b) a heterologous fusion partner protein derived from protein D, wherein the said fusion partner protein does not include the secretion sequence or signal sequence from protein D.

2. A fusion partner protein derived from protein D, in which the fusion partner protein comprises amino acids Met-Asp-Pro at or within the N-terminus of the fusion protein sequence and in which the fusion partner protein does not include the secretion sequence or signal sequence of protein D.

3. The fusion partner protein of claim 2, in which the protein D sequence comprises or consists of approximately or exactly amino acids 17 to 127, 18 to 127, 19 to 127 or 20 to 127 of protein D.

4. The fusion partner protein of claim 1 in which one or more amino acids from the protein D fusion partner protein are deleted or replaced by substitution.

5. The fusion partner protein of claim 4 in which the amino acids are substituted with conservative substitutions.

6. The fusion partner protein of claim 4 in which 1, 2, 3, 4, 5, 6, 7, 8, 9 or more amino acids are substituted.

7. The fusion partner protein of claim 1 in which the secretion sequence or signal sequence of protein D refers to approximately amino acids 1 to 16, 17, 18 or 19 of the naturally occurring protein.

8. The fusion partner protein of claim 1 in which the secretion or signal sequence of protein D is the N-terminal 19 amino acids of protein D.

9. The fusion protein comprising the fusion partner protein of claim 2.

10. The fusion protein comprising the fusion partner protein of claim 2 and one or more tumour antigens or immunogenic portions thereof.

11. The fusion protein of claim 9 comprising the tumour antigen PRAME or an immunogenic portion thereof.

12. The fusion protein of claim 1 in which the immunogenic fragment or portion of PRAME comprises or consists of one or more of the following epitopes:

| | |
|---|---|
| VLDGLDVLL; | (PRA¹⁰⁰⁻¹⁰⁸; SEQ ID NO: 13) |
| SLYSFPEPEA; | (PRA¹⁴²⁻¹⁵¹; SEQ ID NO: 14) |
| ALYVDSLFFL; | (PRA³⁰⁰⁻³⁰⁹; SEQ ID NO: 15) |
| LYVDSLFFL | (PRA³⁰¹⁻³⁰⁹; SEQ ID NO: 16) |
| and | |
| SLLQHLIGL. | (PRA⁴²⁵⁻⁴³³; SEQ ID NO: 17) |

13. The fusion protein of claim 10 comprising one or more tumour antigen selected from the group consisting of
MAGE 1, MAGE 2, MAGE 3, MAGE 4, MAGE 5, MAGE 6, MAGE 7, MAGE 8, MAGE 9, MAGE 10, MAGE 11, MAGE 12, MAGE B1, MAGE B2, MAGE B3, MAGE B4, MAGE C1, MAGE C2.
or a tumour antigen derivative or an immunogenic portion thereof.

14. The fusion protein of claim 13 in which 1, 2, 3, 4, 5, 6, 7, 8, 9, 10 or more amino acids may be deleted from or substituted in the amino acid sequence of the MAGE antigen.

15. The fusion protein of claim 14 in which 2 amino acids are deleted from the N-terminus of the MAGE sequence.

16. The fusion protein of claim 15 wherein the antigen is MAGE-A3 or an immunogenic portion thereof, in which the MAGE-A3 antigen comprises or consists of amino acid 3 to 314 of MAGE-A3.

17. The fusion protein of claim 10 in which the tumour antigen or derivative thereof is selected from one of the following antigens or an immunogenic portion thereof which is able to direct an immune response to the antigen: WT-1. WT-1F, BAGE, LAGE 1, LAGE 2 (also known as NY-ESO-1), SAGE, HAGE, XAGE, PSA, PAP, PSCA, P501S (also known as prostein), HASH1, HASH2, Cripto, B726, NY-BR1.1, P510, MUC-1, Prostase, STEAP, tyrosinase, telomerase, survivin, CASB616, P53, and/or Her-2/neu, SSX-2; SSX-4; SSX-5; NA17; MELAN-A; P790; P835; B305D; B854; CASB618 (as described in WO00/53748); CASB7439 (as described in WO01/62778); C1491; C1584; and C1585.

18. The fusion protein of claim 1, further comprising an affinity tag.

19. The fusion protein of claim 1 additionally comprising one or more linker sequences between the fusion partner protein and the tumour antigen or immunogenic portion thereof; and/or between the fusion partner protein and a His tail or other affinity tag; and/or between the tumour antigen or immunogenic portion thereof and a His tail or other affinity tag.

20. A nucleic acid molecule comprising a nucleic acid sequence encoding the fusion protein or fusion partner protein of claim 1.

21. The nucleic acid molecule of claim 20 further comprising a vector.

22. A host cell transformed with the nucleic acid molecule of claim 21.

23. A vaccine comprising:
(a) the nucleic acid molecule of claim 20; or
(b) the fusion protein or fusion partner protein encoded by the nucleic acid molecule of claim 20.

24. The vaccine of claim 23 additionally comprising one or more components selected from the group consisting of: adjuvant, immunostimulatory cytokine, and chemokine.

25. The vaccine of claim 24 wherein the adjuvant comprises one or more components selected from the group consisting of: 3D-MPL, QS21, and CpG oligonucleotide.

26. A method for inducing an immune response in a mammal comprising the step of administering to the mammal the vaccine of claim 23.

27. (canceled)

28. A process for producing a fusion protein comprising the step of inducing the host cell of claim 22 to express the fusion protein encoded by the nucleic acid molecule therein.

29. The process of claim 28 in which the cell is a bacterium.

30. The process of claim 29 in which the bacterium is E. coli.

31. The process of claim 28 in which the fusion protein is expressed in a cell as an insoluble protein.

32. The process of claim 31 further comprising the step of lysing the cell and purifying the expressed fusion protein from the lysed cells.

33. The fusion protein obtained by or obtainable by the process of claim 28.

34. A method of treating a patient suffering from cancer comprising the step of administering the vaccine of claim 23.

35. The method of claim 34 in which the cancer is selected from the group consisting of melanoma, breast, bladder, lung cancer such as NSCLC, sarcoma, ovarian cancer, head and neck cancer, renal cancer, colorectal carcinoma, multiple myeloma, and leukemia including acute leukemia and oesophageal carcinoma.

36. The fusion partner protein of claim 2 in which one or more amino acids from the protein D fusion partner protein are deleted or replaced by substitution.

37. The fusion partner protein of claim 36 in which the amino acids are substituted with conservative substitutions.

38. The fusion partner protein of claim 37 in which 1, 2, 3, 4, 5, 6, 7, 8, 9 or more amino acids are substituted.

39. The fusion partner protein of claim 2 in which the secretion sequence or signal sequence of protein D refers to approximately amino acids 1 to 16, 17, 18 or 19 of the naturally occurring protein.

40. The fusion partner protein of claim 2 in which the secretion or signal sequence of protein D is the N-terminal 19 amino acids of protein D.

41. The fusion protein of claim 11 in which the immunogenic fragment or portion of PRAME comprises or consists of one or more of the following epitopes:

| | |
|---|---|
| VLDGLDVLL; | (PRA¹⁰⁰⁻¹⁰⁸; SEQ ID NO: 13) |
| SLYSFPEPEA; | (PRA¹⁴²⁻¹⁵¹; SEQ ID NO: 14) |
| ALYVDSLFFL; | (PRA³⁰⁰⁻³⁰⁹; SEQ ID NO: 15) |
| LYVDSLFFL | (PRA³⁰¹⁻³⁰⁹; SEQ ID NO: 16) |
| and | |
| SLLQHLIGL. | (PRA⁴²⁵⁻⁴³³; SEQ ID NO: 17) |

42. The fusion protein of claim 2, further comprising an affinity tag.

43. The fusion protein of claim 2 additionally comprising one or more linker sequences between the fusion partner protein and the tumour antigen or immunogenic portion thereof; and/or between the fusion partner protein and a His tail or other affinity tag; and/or between the tumour antigen or immunogenic portion thereof and a His tail or other affinity tag.

44. A nucleic acid molecule comprising a nucleic acid sequence encoding the fusion protein or fusion partner protein of claim 2.

45. The nucleic acid molecule of claim 44 further comprising a vector.

46. A host cell transformed with the nucleic acid molecule of claim 45.

47. A vaccine comprising:
(a) the nucleic acid molecule of claim 44; or
(b) the fusion protein or fusion partner protein encoded by the nucleic acid molecule of claim 44.

48. The vaccine of claim 47 additionally comprising one or more components selected from the group consisting of: adjuvant, immunostimulatory cytokine, and chemokine.

49. The vaccine of claim 48 wherein the adjuvant comprises one or more components selected from the group consisting of: 3D-MPL, QS21 and/or a CpG oligonucleotide.

50. A method for inducing an immune response in a mammal comprising the step of administering to the mammal the vaccine of claim 48.

51. A process for producing a fusion protein comprising the step of inducing the host cell of claim 46 to express the fusion protein encoded by the nucleic acid molecule therein.

52. The process of claim 51 in which the cell is a bacterium.

53. The process of claim 52 in which the bacterium is E. coli.

54. The process of claim 51 in which the fusion protein is expressed in a cell as an insoluble protein.

55. The process of claim 54 further comprising the step of lysing the cell and purifying the expressed fusion protein from the lysed cells.

56. A fusion protein obtained by or obtainable by the process of claim 51.

57. A method of treating a patient suffering from cancer comprising the step of administering the vaccine of claim 47.

58. The method of claim 57 in which the cancer is selected from the group consisting of melanoma, breast, bladder, lung cancer such as NSCLC, sarcoma, ovarian cancer, head and neck cancer, renal cancer, colorectal carcinoma, multiple myeloma, and leukemia including acute leukemia and oesophageal carcinoma.
